SEQUENCE LISTING (9 sequences)

SEQ ID NO: 1 - DNA, 1528 bp, Papaver somniferum
  CDS (1) ..(1464)
atggagatcgtcacagtatcacttgtagcagttgtgatcactactttcttatacttaatcttcagagattcaagtcctaaaggtttgccaccaggtccaaaaccctggccaatagttggaaaccttcttcaacttggtgagaaacctcattctcagtttgctcagcttgctgaaacctatggtgatctcttttcactgaaactaggaagtgaaacggttgttgtagcttcaactccattagcagctagcgagattctaaagacgcatgatcgtgttctctctggtcgatacgtgtttcaaagtttccgggtaaaagaacatgtggagaactctattgtgtggtctgaatgtaatgaaacatggaagaaactgcggaaagtttgtagaacggacctttttacgcagaagatgattgaaagtcaagctgaagttagagaaagtaaggctatggaaatggtggagtatttgaagaaaaatgtaggaaatgaagtgaaaattgctgaagttgtatttgggacgttggtgaatatattcggtaacttgatattttcacaaaatattttcaagttgggtgatgaaagtagtggaagtgtagaaatgaaagaacatctatggagaatgctggaattggggaactcgacaaatccagctgattattttccatttttgggtaaattcgatttgtttggacaaagcaaagatgttgctgattgtctgcaagggatttatagtgtttggggtgctatgctcaaagaaagcaaaatagccaagcagcataacaacagcaagaagaatgattttgttgagattttgctcgattccggactcgatgaccagcagattaatgccttgctcatggaaatatttggtgcgggaacagagacaagtgcatctacaatagaatgggcgttgtctgagctcacaaaaaaccctcaagtaacagccaatatgcggttggaattgttatctgtggtagggaagaggccggttaaggaatccgacataccaaacatgccttatcttcaagcttttgttaaagaaactctacggcttcatccagcaactcctctgctgcttccacgtcgagcacttgagacctgcaaagttttgaactatacgatcccgaaagagtgtcagattatggtgaacgcctggggcattggtcgggatccaaaaaggtggactgatccattgaagttttcaccagagaggttcttgaattcgagcattgatttcaaagggaacgacttcgagttgataccatttggtgcagggagaaggatatgtcctggtgtgcccttggcaactcaatttattagtcttattgtgtctagtttggtacagaattttgattggggattaccgaagggaatggatcctagccaactgatcatggaagagaaatttgggttgacactgcaaaaggaaccacctctgtatattgttcctaaaactcgggattaatctcaatcaagaaatcttaaattcacatgattatggatttctcgctactttatttgaaaaaaaa

SEQ ID NO: 2 - protein, 487 aa, Papaver somniferum
MEIVTVSLVAVVITTFLYLIFRDSSPKGLPPGPKPWPIVGNLLQLGEKPHSQFAQLAETYGDLFSLKLGSETVVVASTPLAASEILKTHDRVLSGRYVFQSFRVKEHVENSIVWSECNETWKKLRKVCRTDLFTQKMIESQAEVRESKAMEMVEYLKKNVGNEVKIAEVVFGTLVNIFGNLIFSQNIFKLGDESSGSVEMKEHLWRMLELGNSTNPADYFPFLGKFDLFGQSKDVADCLQGIYSVWGAMLKESKIAKQHNNSKKNDFVEILLDSGLDDQQINALLMEIFGAGTETSASTIEWALSELTKNPQVTANMRLELLSVVGKRPVKESDIPNMPYLQAFVKETLRLHPATPLLLPRRALETCKVLNYTIPKECQIMVNAWGIGRDPKRWTDPLKFSPERFLNSSIDFKGNDFELIPFGAGRRICPGVPLATQFISLIVSSLVQNFDWGLPKGMDPSQLIMEEKFGLTLQKEPPLYIVPKTRD

SEQ ID NO: 3 - protein, 481 aa, Papaver somniferum
SLVAVVITTFLYLIFRDSSPKGLPPGPKPWPIVGNLLQLGEKPHSQFAQLAETYGDLFSLKLGSETVVVASTPLAASEILKTHDRVLSGRYVFQSFRVKEHVENSIVWSECNETWKKLRKVCRTDLFTQKMIESQAEVRESKAMEMVEYLKKNVGNEVKIAEVVFGTLVNIFGNLIFSQNIFKLGDESSGSVEMKEHLWRMLELGNSTNPADYFPFLGKFDLFGQSKDVADCLQGIYSVWGAMLKESKIAKQHNNSKKNDFVEILLDSGLDDQQINALLMEIFGAGTETSASTIEWALSELTKNPQVTANMRLELLSVVGKRPVKESDIPNMPYLQAFVKETLRLHPATPLLLPRRALETCKVLNYTIPKECQIMVNAWGIGRDPKRWTDPLKFSPERFLNSSIDFKGNDFELIPFGAGRRICPGVPLATQFISLIVSSLVQNFDWGLPKGMDPSQLIMEEKFGLTLQKEPPLYIVPKTRD

SEQ ID NO: 4 - DNA, 1511 bp, Papaver somniferum
atcacttgtagcagttgtgatcactactttcttatacttaatcttcagagattcaagtcctaaaggtttgccaccaggtccaaaaccctggccaatagttggaaaccttcttcaacttggtgagaaacctcattctcagtttgctcagcttgctgaaacctatggtgatctcttttcactgaaactaggaagtgaaacggttgttgtagcttcaactccattagcagctagcgagattctaaagacgcatgatcgtgttctctctggtcgatacgtgtttcaaagtttccgggtaaaagaacatgtggagaactctattgtgtggtctgaatgtaatgaaacatggaagaaactgcggaaagtttgtagaacggacctttttacgcagaagatgattgaaagtcaagctgaagttagagaaagtaaggctatggaaatggtggagtatttgaagaaaaatgtaggaaatgaagtgaaaattgctgaagttgtatttgggacgttggtgaatatattcggtaacttgatattttcacaaaatattttcaagttgggtgatgaaagtagtggaagtgtagaaatgaaagaacatctatggagaatgctggaattggggaactcgacaaatccagctgattattttccatttttgggtaaattcgatttgtttggacaaagcaaagatgttgctgattgtctgcaagggatttatagtgtttggggtgctatgctcaaagaaagcaaaatagccaagcagcataacaacagcaagaagaatgattttgttgagattttgctcgattccggactcgatgaccagcagattaatgccttgctcatggaaatatttggtgcgggaacagagacaagtgcatctacaatagaatgggcgttgtctgagctcacaaaaaaccctcaagtaacagccaatatgcggttggaattgttatctgtggtagggaagaggccggttaaggaatccgacataccaaacatgccttatcttcaagcttttgttaaagaaactctacggcttcatccagcaactcctctgctgcttccacgtcgagcacttgagacctgcaaagttttgaactatacgatcccgaaagagtgtcagattatggtgaacgcctggggcattggtcgggatccaaaaaggtggactgatccattgaagttttcaccagagaggttcttgaattcgagcattgatttcaaagggaacgacttcgagttgataccatttggtgcagggagaaggatatgtcctggtgtgcccttggcaactcaatttattagtcttattgtgtctagtttggtacagaattttgattggggattaccgaagggaatggatcctagccaactgatcatggaagagaaatttgggttgacactgcaaaaggaaccacctctgtatattgttcctaaaactcgggattaatctcaatcaagaaatcttaaattcacatgattatggatttctcgctactttatttgaaaaaaaa

SEQ ID NO: 5 - DNA, 3816 bp, Papaver somniferum
aaatttgtacctattacgtctcttctttctggtataccaggcagtggctatcttagtcatgcgattaacttgtacctccgctgtaagttctttactgtggatttgaaacgaattaaaactactttatgtctatccgttccttccatgcttaaattatattctcctcggactcaattgagctgctcatcaacatgtttattatttgacaatgcaaaccgattcaaactagtattctccactaccacttgtagcatgactcattttctggatgtatcgatctctgaatgttcatttatattcatgagaagtgcattgtgtcttcttttctcttttggctattaatattctaaatcatatttggccgttagataaacctgatccaaaggatttgcaatacttcctggagttccaagttccaaggcagtgggcacctatgtttaatgagctgcctcttgggcaccaaaggagggcctcttgtccttctttgcagttcagtttcatgggatcaaagcttcatgtcaactctacgcaggtacactctatttattcttggatatatcttatttttaccaaaatactcttggttgtgctgtactactgtctaaaacaatcctggttgtttagttgtgttttacttaagcgaggtcttatgaagagttccaaacagagtttaggctcgaatagaagatccaaaaacaatagattagaaaaggtgaactaggatatagtagtttttgggtatatcatttcgttttttcatagaacactgagagattggtagaagactgtctcaacgcgaccttaacatgcttgtataacctggctcagagcttgctttatattaacctgcttgttgagtttcaaactttagtcctgtgtgctttcacggcacctgaaacctataataattgttctgaaacaaaataatgttaatacttgtacaaatactgaggctaacattaagtaaatttgttatgttttgagcatattatttaacaaggggttagcagagatagattggagaaaataggcttgatcatatgggtaagagttaggttgattcatcttaacctgtctacattatctcctagccaatgcatataagaaaaaaggatcaaatcgtaattgatttactccatatttcaaataatggttaaaccgacagaacataaggttttataaaggtggtttgcccttctaaatcctgctcccatagataaagctttaagtacaaataccgttttgttaataaaatatagtaataatttgtttttttgctaaactgatgcttttagcatgcttcagaaggcaggctattctaaaatgattttagcatgcttcttaaagcaggctgttaatatttgaagctttcttaagcattgatgccatcataataaaagagggaggtcccattgacattgttaaatttatactatcgacattttttatgctgaagtttttttaatattcaagtgaaaatagtcggaagcttactgttcatatttaacaatcttcttgatgtggtacactctcgcaaaaaattagcgcatttcatgacttgtaccctaactttttaccagtttgaaactcaactgtttgttagcaacaattgaaatcaaaaccagtaaatgatggatacatactacgaaatgcactaatttttggtgggagtggagtgcataatgagaagagcatcatacctaaagaattagattataactagtttttatttgaaccgcataaaaaaactagggtcgaatgtgtaagatagtgtcaatttaacaatgtcaacggaacctctttcatatgtaaagtatcatcacatgataagcaaaacatatatatgacttccggagcctcagtaggatcaatgtagttattaccaatttcaattacattacctggtaggatcacaatcattaatattttaggttaagagcagaatttgtcgtgtatgacttgtattaataaatttttggcgtcaattctaggtctcaagtggcaggactcctgtggtaggtcttcgtctttacctagaagggaaggcatgcaaccgcttggccatacatgtacagcacctctcgaaccttccaaggttacttgaatcttcatgggctgaccccactacgctgaaacaatgtcaatggcgagggtctgatgaatccagtgtcccatacttagaacccataaaatggaaaagatactccaacatttgcacatcagtcgtaaaacatgatccaggttggttgcatggagaatcaccaggtgtattcattgtcactggagctcaactcatgaccaaggggaaatggccaaaacacattcttcaccttcgtttactctacacacatattcctggtttcagcatccagaaaacagaatgggcagttgcaccagcatcttcacaaaaatcaagtttcctcgccaatttcagcacaactttcacgacttcaggtccttcaaaggcagctcctgtggtagctataaattccggagtgtattctgatggccctccaagaccagttcactctcaaaagcttcttaaatatgttgagacgtcagaaatagtccgggggccacacgatattcccggacactggatggtaattgctgctaagctagttacagaaggtggcaagattggtttacttgtaaaatttgcattgcttgattattccagtcccacgagtgagtagaagaaagtttgatccacattggatctcacctaaatctccccatctatcctcctgaagagaagtttgtttcaaactccggttcgaagaataccccatgctaatgtaatgtaagtattataatggatccgtagttattgaagtttttcgaccaactctactttgtcgctggggatagttactataaagctttagaagtaagaaaccgtgtacagaaatctcaaggcctccccctcttcctttttctttttttttttcttctctcattaaatttaatctgtatgtaaattgagaagttatagagtttggttggtggtttcaaataatgtattttcatttaagcttattatgattatatcatctctgagtatcaattccacttgactcttaactgtgaaatcagaacatgacatatgggtgccttgaaaggttattcatatcctgggacttcttgtacatgtaagttgaaaacatcaacttcattcaaagaaattattagaaaaaaccttccaacagtctatctggattagattccatgttgaatcaaccactattttgtacacctacaggcaggctactgttccatatgttagcatgtggatatgcacaatttgacaagttttgtctgttaaaccaaaaattattcaaaaaagttctcccacgcacccccttaaaaaaatgcccacatgcatctcgtgtgtagcacttgacaatccacgagtgccatccaaattgaatttccctacaagattctggacatacttgctctatgaaacttaacttctttatataaacaaccaaagaaaaatacattgagagaaaatatggagatcgtcacagtatcacttgtagcagttgtgatcactactttcttatacttaatcttcagagattcaagtcctaaaggtttgccaccaggtccaaaaccctggccaatagttggaaaccttcttcaacttggtgagaaacctcattctcagtttgctcagcttgctgaaacctatggtgatctcttttcactgaaactaggaagtgaaacggttgttgtagcttcaactccattagcagctagcgagattctaaagacgcatgatcgtgttctctctggtcgatacgtgtttca

SEQ ID NO: 6 - protein, 487 aa, Eschscholzia californica
GTSTVALIAVIISSILYLLFGGSGHKNLPPGPKPWPIVGNLLQLGEKPHAQFAELAQTYGDIFTLKMGTETVVVASTSSAASEILKTHDRILSARYVFQSFRVKGHVENSIVWSDCTETWKNLRKVCRTELFTQKMIESQAHVREKKCEEMVEYLMKKQGEEVKIVEVIFGTLVNIFGNLIFSQNIFELGDPNSGSSEFKEYLWRMLELGNSTNPADYFPMLGKFDLFGQRKEVAECLKGIYAIWGAMLQERKLAKKVDGYKSKNDFVDVCLDSGLNDYQINALLMELFGAGTETSASTIEWAMTELTKNPKITAKIRSEIQTVVGERSVKESDFPNLPYLEATVKETLRLHPPTPLLLPRRALETCTILNYTIPKDCQIMVNAWGIGRDPKTWTDPLTFSPERFLNSSVDFRGNDFSLIPFGAGRRICPGLPIANQFIALLVATFVQNLDWCLPNGMSVDHLIVEEKFGLTLQKEPPLFIVPKSRV

SEQ ID NO: 7 - DNA, 1901 bp, Eschscholzia californica
cggcacgagcacagtagcacttattgcagtaataatttcttcaatactatacctcctctttggtggtagtggtcacaaaaatctcccaccaggaccaaaaccatggccaatagttggaaatctcctccaacttggtgagaaaccacacgctcaattcgccgaactagctcaaacctatggtgacattttcactcttaaaatgggtactgaaactgtagttgttgcatcaacatcttcagcagcttccgaaatactaaaaacccatgatcgaattctatccgctcgttacgtttttcaaagttttcgagtaaaagggcatgtagaaaattcaatagtttggtcagattgtactgaaacttggaagaatttaagaaaagtttgtaggacagaacttttcacacagaagatgatagaaagtcaagctcatgttagagagaaaaaatgtgaagaaatggttgaatacttgatgaaaaaacaaggggaagaagtgaaaattgtggaagtaatatttggaacattagtgaatatatttggaaatttgatattttcacagaatatatttgaattgggtgatccaaatagtggaagttcagagttcaaggaatatctatggaggatgttggaattggggaattcaacaaatccagctgattattttccaatgttaggtaaatttgatttgtttggacagaggaaagaagttgcagagtgtttaaaagggatttatgcaatatggggagctatgcttcaagaaagaaaattagctaaaaaagttgatggatataaaagcaagaatgattttgttgatgtttgtcttgattctggacttaatgattatcaaatcaatgccttgcttatggaattatttggggcaggcacagaaacgagcgcatcgacaattgagtgggccatgactgaactaacaaagaatccaaagataacagctaagattagatcagaaattcaaacagtggtaggcgagagatcggtaaaagaatccgacttccccaatcttccataccttgaagctactgttaaagaaaccctaagacttcacccaccaactccattgctactcccacgccgagcacttgaaacctgtacaatcctcaactataccatcccaaaagattgtcaaattatggtcaacgcttggggaatcggtcgtgatcccaagacttggaccgatccgttgactttctcaccagagagattcttgaattctagtgttgactttagggggaatgatttcagtttgataccatttggtgcaggaagaaggatatgccccggtctgccaatagcaaatcagtttattgcattgctagtggcaacatttgtgcaaaatttggattggtgtctaccaaatgggatgagtgttgaccatttgatagtggaggagaagtttgggttgactcttcaaaaagaaccacctctattcattgttcctaaatcaagggtttgatctttctcatcatggttcatgtgaaaactagattatcttttgttttggctatgcatctgttttctttccttcctataagcttttgttcaccaagtaagaaaatgtccacacgatttttgttcggccgaggtccgagggtatcaagactatggacaacaatcctctgagacaactaaggggtcactggatcaaaattgtcgatatgtacattttcttctgaacaaggttacacattctagctattaggtgtcgcgctaataaaatataataatcacttttgatagttttgtagagacatgttatgcaatacatatctattgcaatacgcatgctcataatgcatatacatatttgaaaagttgtggatgttgttccatgttcaaccataaagcgagtgttttactatgttaaagtgaaaaaaaaaaaaaaaaaaaaa

SEQ ID NO: 8 - protein, 488 aa, Eschscholzia californica
  MISC_FEATURE (192) ..(192): Xaa = unknown
MEVVTVALIAVIISSILYLLFGSSGHKNLPPGPKPWPIVGNLLQLGEKPHAQFAELAQTYGDIFTLKMGTETVVVASTSSAASEILKTHDRILSARYVFQSFRVKGHVENSIVWSDCTETWKNLRKVCRTELFTQKMIESQAHVREKKCEEMVEYLMKKQGEEVKIVEVIFGTLVNIFGNLIFSQNIFELGXPNSGSSEFKEYLWRMLELGNSTNPADYFPMLGKFDLFGQRKEVAECLKGIYAIWGAMLQERKLAKKVDGYQSKNDFVDVCLDSGLNDYQINALLMELFGAGTETSASTIEWAMTELTKNPKITAKLRSELQTVVGERSVKESDFPNLPYLEATVKETLRLHPPTPLLLPRRALETCTILNYTIPKDCQIMVNAWGIGRDPKTWIDPLTFSPERFLNSSVDFRGNDFSLIPFGAGRRICPGLPIANQFIALLVATFVQNLDWCLPNGMSVDHLIVEEKFGLTLQKEPPLFIVPKSRV

SEQ ID NO: 9 - DNA, 1613 bp, Papaver somniferum
  misc_feature (612) ..(612): N = unknown
aagtccgagtgagaagcagagttagagaaaaaaaaaatggaggttgtcacagtagcacttattgcagtaataatttcttcaatactctacctcctctttggtagtagtggtcacaaaaatctcccaccaggaccaaaaccatggccaatagtaggaaatcttctccaacttggtgagaaaccacacgctcaattcgccgaactagctcaaacctatggtgacattttcactcttaaaatgggtactgaaactgtagttgttgcatcaacatcttcggcagcttccgaaatactaaaaacccatgatcgaattctatccgctcgttacgtttttcaaagttttcgagtaaaagggcatgtagaaaattcaatagtttggtcagattgtactgaaacttggaagaatttaagaaaagtttgtaggacggaacttttcacacagaagatgatagaaagtcaagctcatgttagagagaaaaaatgtgaagaaatggttgaatacttgatgaaaaaacaaggggaagaagtgaaaattgtggaagtaatatttggaacattagtgaatatattcggaaatttgatattttcacagaatatatttgaattgggtganccaaatagtggaagttcagagttcaaggaatatctatggaggatgttggaattagggaattcaacaaatccagctgattattttccaatgttgggtaaatttgatttgtttggacagaggaaagaagttgcagagtgtttaaaagggatttatgctatttggggagctatgcttcaagaaaggaaattagctaaaaaagttgatggataccaaagcaagaatgattttgttgatgtttgtcttgattctggacttaatgattatcagatcaatgccttgcttatggaattatttggggcaggcacagaaacaagcgcatcgacaattgagtgggccatgactgaactaacaaagaatccaaagataacagctaagcttagatcagaacttcaaacagtggtaggcgagagatcggtaaaagaatccgacttccccaatcttccataccttgaagctactgttaaagaaaccctaagacttcacccaccaactccattgctactcccacgtcgagcacttgaaacctgtacaatcctcaactacaccatcccaaaagattgtcaaattatggtcaacgcttggggaatcggacgtgatcccaagacttggatcgatccgttgactttctcaccagagagattcttgaattctagtgttgactttagggggaatgatttcagtttgataccatttggtgcaggaagaaggatatgccccggtctgccaatagcaaatcagtttattgcattgctagtggcaacatttgtgcaaaatttggattggtgtctaccaaatgggatgagtgttgaccatttgatagtggaggagaagtttgggttgactcttcaaaaagaaccacctctattcattgttcctaaatcaagggtttgatcttctcatcatggttcatgtgaaaactagattatctattgtgaaggctatgtatgttttctttccttcctataaaaaaaaaaaaaaaaaaaaaaaaaaaaaaaaaaaaa

(19) United States
(12) Patent Application Publication
(10) Pub. No.: US 2008/0196123 A1
(43) Pub. Date: Aug. 14, 2008

(54) Modulation of Alkaloid Siosynthesis in Plants and Plants Having Altered Alkaloid Biosynthesis

(76) Inventors: Toni M. Kutchan, St. Louis, MO (US); Susanne Frick, Augsberg (DE); Katja Kempe, Chemnitz (DE)

Correspondence Address: THOMPSON COBURN, LLP, ONE US BANK PLAZA, SUITE 3500, ST LOUIS, MO, 63101, US

(21) Appl. No.: 11/660,119

(22) PCT Filed: Aug. 11, 2005
(86) PCT No.: PCT/EP2005/009232

§ 371 Date: Mar. 12, 2008

(30) Foreign Application Priority Data
Aug. 12, 2004 (EP) 04019154.6

(51) Int. Cl. C07D 489/10; C12N 15/52; A01H 5/00

(52) U.S. Cl. 800/286; 800/278; 800/294; 800/298; 536/23.2; 546/46; 546/45; 546/44

(57) ABSTRACT

The invention relates to a method for altering alkaloid biosynthesis in a plant, comprising: i) introducing into cells of a plant, an expressible exogenous nucleic acid comprising or consisting of an (S)-N-methylcoclaurine 3′-hydroxylase gene (cyp80b) or a derivative thereof, and ii) optionally propagating the plant, wherein expression of the exogenous nucleic acid in the plant or in its progeny results in altered levels of alkaloid biosynthesis.

The present invention relates to methods of modulating alkaloid biosynthesis in plants, particularly tetrahydrobenzylisoquinoline-derived alkaloids. The invention also relates to methods for producing plants having altered alkaloid biosynthesis and to methods of producing alkaloids from these plants. The invention also concerns nucleic acid molecules capable of altering alkaloid biosynthesis in plants.

Alkaloids are physiologically active, nitrogen-containing low-molecular weight compounds produced predominantly in higher plants. The tetrahydrobenzylisoquinoline-derived alkaloids include a vast number of structurally diverse molecules that vary widely in physiological activity. The muscle relaxant (+) tubocurarine, the narcotic analgesic morphine, the antitussive and analgesic codeine, and the antimicrobial berberine are examples of this group of alkaloids.

Amongst the alkaloid-producing plants, Papaver somniferum L. is one of the most important. It is considered to be one of the oldest cultivated medicinal plants of Europe. Opium poppy originated in the eastern Mediterranean and the ancient Sumerians used its seed for food. The “sleep-inducing” property and the medicinal value of the latex have also been known and used throughout human history (Brownstein, 1983; Husain and Sharma, 1983). P. somniferum contains more than eighty tetrahydrobenzylisoquinoline-derived alkaloids, including morphine, and codeine, as well as the muscle relaxant papaverine, the antitumoric agent noscapine (Ye et al., 1998) and the antimicrobial sanguinarine. Although morphine biosynthesis is well understood at the enzymic level (reviewed in Kutchan, 1998), the regulation and ecological function of the morphinan alkaloids in planta is still unknown.

The opium poppy has been selected to produce three types of plants: ornamentals, narcotic cultivars and condiment/oilseed cultivars. The pharmaceutical and chemical uses of P. somniferum could be optimized and tailored to specific requirements if the alkaloid metabolism could be altered in a controlled manner. For example poppy seed oil finds use in the chemical industry for the production of pigments and lacquer, but its residual morphine levels prevents more widespread applications. Similarly, it could be desirable to optimise the production of certain alkaloids, such as thebaine, which are less likely to abuse by drug traffickers than morphine, the precursor of heroin, and yet remain useful for the production of therapeutically active derivatives.

With genetic transformation it may be possible to alter the alkaloid metabolism in commercial poppy cultivars in order to obtain varieties lacking alkaloids or to produce plants with alkaloid profiles designed for specific industrial and pharmaceutical uses. However, whilst a number of genes encoding enzymes involved in the biosynthetic pathways of the alkaloid-producing plants have now been isolated, metabolic engineering of these plants is hampered by the lack of knowledge of the regulatory mechanisms governing the pathways, including the involvement of potentially rate-limiting factors such as cytochrome P-450 and compensatory feedback mechanisms which may come into play in the plant.

Thus whilst the molecular tools now available should theoretically permit modifications to be made in the activity of enzymes acting at some steps of the alkaloid biosynthetic pathways, such changes may not necessarily lead to modification of alkaloid profiles. This is especially true if the enzymatic steps in question are upstream of a branch point in the pathway, and if changes are sought in one pathway in preference to another.

It is thus an object of the present invention to identify means of modifying alkaloid profiles in plants, particularly the tetrahydrobenzylisoquinoline-derived alkaloids. It is also an object of the invention to identify means of modifying the levels of morphinan alkaloids produced by plants such as P.somniferum.

The present invention meets these objectives. It has surprisingly been found that expression of the enzyme (S)-N-methylcoclaurine 3′-hydroxylase appears to limit the ability of plants such as P.somniferum to synthesize alkaloid, and that modulation of the expression of this enzyme has a marked effect on alkaloid production levels.

(S)-N-methylcoclaurine 3′-hydroxylase is a cytochrome P-450-dependent monooxygenase. It catalyses hydroxylation of (S)-N-methylcoclaurine to (S)-3′-hydroxy-N-methylcoclaurine on the pathway to the branchpoint isoquinoline alkaloid intermediate (S)-reticuline (FIG. 1). Subsequent stereo- and regio-specific oxidation of (S)-reticuline determines which class of alkaloids will be formed, depending upon whether the morphine-, laudanine- or sanguinarine biosynthetic pathways are followed. It is thus common to these three biosynthetic pathways of P. somniferum alkaloids.

(S)-N-methylcoclaurine 3′-hydroxylase is a member of the cytochrome P450 super-family and is classified in the CYP80 family, more particularly the CYP80B sub-family. Standard cytochrome-P450 classification is based on primary amino acid sequence. A family normally comprises those P450s sharing over 40% amino acid identity, and a sub-family generally groups together enzymes showing 55% or more identity. The CYP80B sub-family includes (S)-N-methylcoclaurine 3′-hydroxylase enzymes from different plant species. For example, at present, the enzymes from Eschscholzia californica (California poppy, Ranunculales), Coptis japonica (Japanese goldthread, Ranunculales), Papaver somniferum (opium poppy, Ranunculales) and Thalictrum flavum (yellow meadow rue, Ranunculales) are included in this sub-family and, according to recent nomenclature proposals, may be designated as CYP80B1, CYP80B2, CYP80B3 and CYP80B4 respectively, the corresponding genes being designated cyp80b1, cyp80b2, cyp80b3 and cyp80b4. In the present application, the designation CYP80B1, unless otherwise specified, is used as a general designation for (S)-N-methylcoclaurine 3′-hydroxylase enzymes from any species. Correspondingly, in the present application, unless otherwise indicated, the designation cyp80b1 signifies a gene encoding (S)-N-methylcoclaurine 3′-hydroxylase enzymes from any species.

The cDNA of the cyp80b1 gene encoding (S)-N-methylcoclaurine 3′-hydroxylase in E. californica (CYP80B1) has been isolated (Pauli and Kutchan, 1998), as has partial cDNA of a P.somniferum gene (Huang and Kutchan, 2000).

The present inventors have determined that up-regulation of the cyp80b1 gene encoding (S)-N-methylcoclaurine 3′-hydroxylase leads to significantly increased total levels of alkaloids in latex. Correspondingly, down-regulation of the gene leads to reduced levels. This finding is unexpected for several reasons. First, the enzyme (S)-N-methylcoclaurine 3′-hydroxylase is cytochrome P-450-dependent, and thus the effects of its over-expression are dependent on availability of cytochrome P-450. It is noteworthy that the modulatory effects of the present invention are obtained without the need to cause accumulation of higher levels of cytochrome P-450 in the transgenic tissue, such as by concomitant over-expression of P450 reductase. Moreover, the metabolic step catalysed by (S)-N-methylcoclaurine 3′-hydroxylase is many steps upstream of the end products of the pathways, particularly the morphinan pathway. Since the number of downstream steps is high, the probability that one of these steps might be rate-limiting, thereby neutralising the effects of over-expression of (S)-N-methylcoclaurine 3′-hydroxylase was significant. This however does not appear to be the case. Furthermore, it could not be excluded that inhibitory feed-back mechanisms might compensate for over- or under-expression of (S)-N-methylcoclaurine 3′-hydroxylase, preventing changes in alkaloid production.

The present invention thus concerns a method for altering alkaloid biosynthesis in a plant, said method comprising modifying the expression of an (S)-N-methylcoclaurine 3′-hydroxylase gene in the plant. The levels of the enzyme (S)-N-methylcoclaurine 3′-hydroxylase produced by the plant are thus increased or decreased, thereby increasing or decreasing the levels of alkaloids produced.

According to the invention the modification of the expression of an (S)-N-methylcoclaurine 3′-hydroxylase gene can comprise induction, enhancement, suppression or inhibition of expression of endogenous (S)-N-methylcoclaurine 3′-hydroxylase gene sequences in the plant. It may also comprise modification in the spatio-temporal expression of the enzyme.

Alteration of (S)-N-methylcoclaurine 3′-hydroxylase gene expression can be achieved using any of the known techniques for gene regulation. For example, expression can be up-regulated by increasing the copy number of the endogenous gene using amplification techniques. One way of amplifying the gene is for example to insert genes encoding amplifying agents such as DHFR in the vicinity of the gene in the plant genome. Alternatively, the copy number can be increased by introducing exogenous copies of the gene using recombinant techniques. Expression can be down-regulated by use of gene-silencing techniques such as antisense suppression, sense co-suppression, RNA interference, ribozymes, DNAzymes etc. More generally, (S)-N-methylcoclaurine 3′-hydroxylase gene expression can be changed by modifying the regulatory sequences controlling transcription of the gene, using for example homolgous recombination to introduce or inactivate specific sequences in a targeted manner. For example, endogenous regulatory sequences can be replaced, activated, inactivated or multiplied, enabling a vast range of effects on gene expression to be achieved. A further technique allowing modification of the (S)-N-methylcoclaurine 3′-hydroxylase gene expression is mutation.

According to a preferred embodiment of the invention, the alteration of (S)-N-methylcoclaurine 3′-hydroxylase gene expression, and the concomitant alteration in alkaloid biosynthesis, is achieved by introducing into the plant, nucleic acid sequences derived from the (S)-N-methylcoclaurine 3′-hydroxylase gene.

More specifically, this aspect of the invention concerns a method for altering alkaloid biosynthesis in a plant, comprising:
i) introducing into cells of a plant, an expressible exogenous nucleic acid comprising or consisting of an (S)-N-methylcoclaurine 3′-hydroxylase gene, or a derivative thereof, and
ii) optionally propagating the plant,
   wherein expression of the exogenous nucleic acid in the plant or in its progeny results in altered levels of alkaloid biosynthesis.

According to this embodiment of the invention, the exogenous nucleic acid may be any (S)-N-methylcoclaurine 3′-hydroxylase gene or derivative thereof.

Depending on the precise nature of the exogenous nucleic acid, its expression in the plant will give rise either to over-expression of (S)-N-methylcoclaurine 3′-hydroxylase, or to decreased or abolished expression of (S)-N-methylcoclaurine 3′-hydroxylase. It is also possible to achieve a shift in the spatial or temporal expression of the enzyme.

According to a first variant of the invention, the exogenous nucleic acid is a (S)-N-methylcoclaurine 3′-hydroxylase gene, particularly a (S)-N-methylcoclaurine 3′-hydroxylase gene of a plant belonging to the order Ranunculales. Typical examples are the cyp80b1 gene of Papaver somniferum, encoding the protein illustrated in FIG. 6 (SEQ. ID. N⁰ 1 and SEQ. ID. N^{o} 2), or the cyp80b1 gene of Eschscholzia californica, encoding proteins illustrated in FIGS. 10 and 12 (SEQ. ID. N^{o} 6 and SEQ. ID. N^{o} 8 respectively). Other examples are genes encoding other (S)-N-methylcoclaurine 3′-hydroxylase enzymes of the CYP80B sub-family, showing at least 55% identity in amino acid sequence with those illustrated in FIGS. 6 (SEQ. ID. N^{o} 2), 10 (SEQ. ID. N^{o} 6)and 12 (SEQ. ID. N^{o} 8), for example at least 70% identity. The genes may be the naturally occurring genes (genomic or cDNA) or may be altered in some way, composed for example of a coding sequence, together with heterologous transcription regulatory sequences. The coding sequences may be full length or truncated and generally encode an enzymatically active protein.

Use of such “sense” gene sequences as the exogenous nucleic acid of the invention gives rise, predominantly, to an over-expression of (S)-N-methylcoclaurine 3′-hydroxylase, and to a corresponding increase in the alkaloid levels produced by the plant. For over-expression of the enzyme using sense sequences it is preferred that the coding sequence be full length. It is also possible to obtain a co-suppression effect with the gene “sense” sequences.

The exogenous nucleic acid may also be a derivative of a (S)-N-methylcoclaurine 3′-hydroxylase gene. In this context, the term “derivative” signifies any sequence which is derived from the gene or its cDNA or its transcript, for example by fragmenting, copying, amplifying, transcribing, reverse transcribing, splicing, subjecting to polymerase, mutating, or by deleting, substituting, inserting, or chemically modifying one or more nucleotides of the gene or cDNA or transcript, or by fusing to heterologous sequences. According to the invention the derivatives have the capacity to exert a modulatory effect on the expression of the gene.

Examples of derivatives of the (S)-N-methylcoclaurine 3′-hydroxylase gene include single or double stranded sequence variants, fragments, splice variants, complementary sequences, RNA equivalents, mixed DNA/RNA equivalents, chemical analogues of said gene. Particularly preferred examples include sense and antisense sequence, ribozyme sequences, DNAzyme sequences, RNA-interference sequences, and sequences capable of giving rise to any of the foregoing or their complements.

Particularly preferred derivatives can be obtained from the cyp80b1 gene of Papaver somniferum. The coding sequence of this gene is illustrated in FIG. 6 (SEQ. ID. N^{o} 1).

More specifically, derivatives of the cyp80b1 gene of Papaver somniferum are chosen from
i) a sequence encoding the protein sequence illustrated in FIG. 6 (SEQ. ID. N^{o} 1),
ii) a variant of the nucleotide sequence illustrated in FIG. 6 (SEQ. ID. N^{o} 1), said variant having at least 70% identity with the sequence of FIG. 6 (SEQ. ID. N^{o} 1 )over a length of at least 1000 bases, and encoding a (S)-N-methylcoclaurine 3′-hydroxylase, or
iii) a fragment of sequence (i) or (ii), said fragment having a length of at least 20 nucleotides, or
iv) a sequence complementary to any one of sequences (i), (ii) or (iii), and having a length of at least 20 nucleotides, or
v) any one of sequences (i), (ii), (iii) or (iv) in double-stranded form, or
vi) the RNA equivalent of any of sequences (i), (ii), (iii), (iv) or (v), or
vii) a combination of any of the foregoing.

In accordance with the first group of derivatives (i), as defined above, the exogenous nucleic acid may comprise any sequence encoding the protein sequence illustrated in FIG. 6 (SEQ. ID. N^{o} 2), including all sequences arising from the degeneracy of the genetic code, particularly the nucleic acid sequence illustrated in FIG. 6 (SEQ. ID. N^{o} 1).

In accordance with the second group of derivatives (ii), as defined above, the exogenous nucleic acid may comprise sequence variants of the nucleotide sequence illustrated in FIG. 6 (SEQ. ID. N^{o} 1). Such variants may or may not code for a protein having (S)-N-methylcoclaurine 3′-hydroxylase activity. The sequence variant has at least 70% identity and preferably al least 85% identity with the nucleotide sequence of FIG. 6 (SEQ. ID. N^{o} 1) over a length of at least 1000 bases, and differs from the sequence of FIG. 6 by insertion, replacement and/or deletion of at least one nucleotide, for example at least 10 nucleotides, up to around 150 nucleotides. Examples of this type of variant include sequences corresponding to the coding sequences of cyp80b1 genes from plant species other than P.somniferum, for example those of E.californica as illustrated in FIG. 11 (SEQ. ID. N^{o} 7) or FIG. 13 (SEQ. ID. N^{o} 9) or portions thereof.

Generally speaking the variants have the capacity to hybridise to the nucleotide sequence illustrated in FIG. 6 (SEQ. ID. N^{o} 1), or its complement, in stringent conditions. Stringent conditions are for example those set out in Sambrook et al., Molecular Cloning: A Laboratory Manual, Cold Spring Harbor Laboratories, Cold Spring Harbor, N.Y., USA, 1989 (Chapter 9, pages 9.47 to 9.57). Generally, as indicated in Sambrook supra, hybridization of nucleic acids having more than 200 nucleotides should be carried out at 15 to 25° C. below the calculated melting temperature Tm of a perfect hybrid. An example of stringent hybridization conditions is hybridization at 4×SSC at 65° C., followed by washing in 0.1×SSC at 65° C. for an hour. Alternatively, an exemplary stringent hybridization condition is in 50% formamide, 4×SSC at 42° C.

Particularly preferred variants have at least 99% identity with the nucleotide sequence of FIG. 6 (SEQ. ID. N^{o} 1) over a length of at least 1000 bases, and differ from the sequence of FIG. 6 by insertion, replacement and/or deletion of at least one nucleotide, for example by one to ten nucleotides, particularly one to five nucleotides. Examples of this type of variant include allelic variants of the cyp80b1 gene of Papaver somniferum, or portions thereof.

Other examples of variants which are suitable for use as exogenous nucleic acids of the invention include hybrid sequences comprising an assembly of portions of cyp80b1 genes from different species, for example a portion of the cyp80b1 gene of P. somniferum and a portion of a cyp80b1 gene of a plant species other than P.somniferum, for example of E.californica. The hybrid sequences may have for example a 5′ portion from a first species, say up to the first 40 amino acids, and the remaining 3′ portion from a second species. Indeed, it has been shown by the inventors that a chimeric coding sequence composed predominantly of the P. somniferum sequence, fused to the N-terminus of the E.californica sequence, is effective in modulating expression of the (S)-N-methylcoclaurine 3′-hydroxylase gene, and of altering the alkaloid profile of P.somniferum.

In accordance with the third group of derivatives (iii), as defined above, the exogenous nucleic acid may comprise or consist of a fragment of a sequence encoding (S)-N-methylcoclaurine 3′-hydroxylase or a fragment of a sequence variant of the (S)-N-methylcoclaurine 3′-hydroxylase gene. Such fragments have a length of at least 20 nucleotides, for example from 23 or 25 nucleotides to around 1400 nucleotides, or from 60 to 500 nucleotides. Short fragments having a length of 20 to 50 nucleotides are particularly useful in the invention for example in generating ribozymes, DNAzymes, interfering RNAs etc.

In accordance with the fourth group of derivatives (iv), as defined above, the exogenous nucleic acid may comprise or consist of a sequence which is complementary to any of the foregoing preferred derivatives. Examples of such sequences include sequences which are complementary to the nucleotide sequence illustrated in FIG. 6 (SEQ. ID. N^{o} 1) or to a part thereof, or to the sequence illustrated in FIG. 11 (SEQ. ID. N^{o} 7) or a part thereof, or to the sequence illustrated in FIG. 13 (SEQ. ID. N^{o} 9) or a part thereof.

In the context of the invention, “complementary” means that Watson-Crick base-pairs can form between a majority of bases in the complementary sequence and the reference sequence. Preferably, the complementarity is 100%, but one or two mismatches in a stretch of twenty or thirty bases can be tolerated. Additionally, complementary stretches may be separated by non-complementary stretches.

In accordance with a preferred embodiment of the invention the derivative comprises an antisense sequence, a ribozyme sequence, a DNAzyme sequence, an RNA-interference sequence, or a sequence capable of giving rise to any of these molecules or their complements. These sequences are generally used to down-regulate or silence expression of the (S)-N-methylcoclaurine 3′-hydroxylase gene, and to consequently give rise to reduced levels of alkaloid production in the plant.

Antisense sequences of the (S)-N-methylcoclaurine 3′-hydroxylase gene may correspond to the full coding sequence, i.e. from the ATG start codon to the stop codon, or may correspond to only part of the coding sequence.

Ribozymes of the invention may be directed to any part of the (S)-N-methylcoclaurine 3′-hydroxylase gene transcript, and may be single or multiple ribozymes. Hammerhead ribozymes are particularly preferred (see EP-A-0 321 201).

DNAzyme sequences i.e. sequences which comprise deoxyribonucleotide bases and have endonuclease activity may also be used according to the invention to inhibit or silence the expression of the (S)-N-methylcoclaurine 3′-hydroxylase gene. Such DNAZymes may be fully DNA or mixed DNA/RNA molecules, and may contain chemically modified bases (see for example International patent application WO96/17086).

Interfering RNA is a further approach which may be used according to the invention to reduce or silence the expression of the (S)-N-methylcoclaurine 3′-hydroxylase gene. Such interference involves generation of short double-stranded RNA molecules essentially similar to part of the nucleotide sequence of the targeted (S)-N-methylcoclaurine 3′-hydroxylase gene (see for example International patent application WO99/05305).

According to the invention, any of the above derivatives may comprise at least one nucleotide analogue in replacement of, or in addition to, a naturally occurring nucleotide. Ribonucleotide and deoxyribonucleotide derivatives or modifications are well known in the art, and are described, for example, in Principles of Nucleic Acid Structure (Ed, Wolfram Sanger, Springer-Verlag, New York, 1984), particularly pages 159-200), and in the CRC Handbook of Biochemistry (Second edition, Ed, H. Sober, 1970). A large number of modified bases are found in nature, and a wide range of modified bases have been synthetically produced. For example, amino groups and ring nitrogens may be alkylated, such as alkylation of ring nitrogen atoms or carbon atoms such as N1 and N7 of guanine and C5 of cytosine; substitution of keto by thioketo groups; saturation of carbon=carbon double bonds. Bases may be substituted with various groups, such as halogen, hydroxy, amine, alkyl, azido, nitro, phenyl and the like. Examples of suitable nucleotide analogues are listed in Table I below. In accordance with this embodiment of the invention, synthetic genes comprising one or more nucleotide analogues, for example methylated bases, are made, for example by chemical synthesis, and can be introduced into cells for a transient expression process in vivo.

In accordance with the invention, the plant whose alkaloid production is altered is a plant which naturally has the capacity to produce endogenous benzylisoquinoline-derived alkaloids. Such plants contain at least one copy of an endogenous (S)-N-methylcoclaurine 3′-hydroxylase gene. Plants belonging to the order Ranunculales are examples of such plants, particularly plants belonging to any one of the families Papaveraceae, Euphorbiaceae, Berberidaceae, Fumariaceae or Ranunculaceae. A particularly preferred plant belongs to the genus Papaver, for example plants of the species Papaver somniferum, Papaver bracteatum, Papaver setigerum, Papaver orientate, Papaver pseud-orientale, Papaver cylindricum, or Papaver rhoeas. The exogenous (S)-N-methylcoclaurine 3′-hydroxylase gene or derivative thereof, expressed in cells of the plant may be the same as, or may be different from, the endogenous (S)-N-methylcoclaurine 3′-hydroxylase gene of the plant.

Papaver somniferum is the most preferred plant on account of its capacity to produce morphinan alkaloids.

In the context of the invention, the alteration of alkaloid biosynthesis particularly relates to the alteration of tetrahydrobenzylisoquinoline-derived alkaloids, including the alkaloids found in latex such as morphine, codeine, codeinone, thebaine, oripavine, reticuline, (S)-laudanine and laudanosine. Implementation of the methods of the invention affects levels of alkaloids of both the morphine biosynthetic pathway (including morphine, codeine, codeinone, thebaine, oripavine) and the laudanine biosynthetic pathway (including (S)-laudanine and laudanosine).

In particular, the altered alkaloid biosynthesis affects levels of at least one alkaloid selected from the group consisting of morphine, codeine, codeinone, thebaine, oripavine, reticuline, (S)-laudanine and laudanosine. In plants of Papaver the alteration may for example result in an increase or a decrease in the amount of total alkaloid in latex of the plant. Changes involving an increase or decrease, by a factor of at least two or three, in the amount of total alkaloid in latex can be obtained. Using sense sequences, increases in as much as four or five times the total amount of alkaloid in latex have been obtained, and using antisense sequences, a reduction of four to five times the total amount of alkaloids in latex has been observed. Similarly, morphine levels can be increased or decreased by a factor of at least two. Such changes have been observed in normal commercial varieties of P.somniferum and also in elite varieties which already have high alkaloid production levels. The proportions of the individual alkaloids in the latex of the plant may or may not be altered. The method of the invention can thus be used to generate high morphine lines, low morphine lines, high thebaine lines, high codeine lines etc.

Particularly preferred examples result in an increase in the proportion of the morphine levels in the latex of the plant.

TABLE I
Nucleotide Analogues

| Abbreviation | Description |
|---|---|
| ac4c | 4-acetylcytidine |
| chm5u | 5-(carboxyhydroxylmethyl)uridine |
| cm | 2′-O-methylcytidine |
| cmnm5s2u | 5-carboxymethylaminomethyl thiouridine |
| d | dihydrouridine |
| fm | 2′-O-methylpseudouridine |
| galq | β,D-galactosylqueosine |
| gm | 2′-O-methylguanosine |
| I | inosine |
| i6a | N6-isopentenyladenosine |
| m1a | 1-methyladenosine |
| m1f | 1-methylpseudouridine |
| m1g | 1-methy[guanosine |
| ml1 | 1-methylinosine |
| m22g | 2,2-dimethylguanosine |
| m2a | 2-methyladenosine |
| m2g | 2-methylguanosine |
| m3c | 3-methylcytidine |
| m5c | 5-methylcytidine |
| m6a | N6-methyladenosine |
| m7g | 7-methylguanosine |
| mam5u | 5-methylaminomethyluridine |
| mam5s2u | 5-methoxyaminomethyl-2-thiouridine |
| manq | β,D-mannosylmethyluridine |
| mcm5s2u | 5-methoxycarbonylmethyluridine |
| mo5u | 5-methoxyuridine |
| ms2i6a | 2-methylthio-N6-isopentenyladenosine |
| ms2t6a | N-((9-β-ribofuranosyl-2-methylthiopurine-6- |
| | yl)carbamoyl)threonine |
| mt6a | N-((9-β-ribofuranosylpurine-6-yl)N-methyl- |
| | carbamoyl)threonine |
| mv | uridine-5-oxyacetic acid methylester |
| o5u | uridine-5-oxyacetic acid (v) |
| osyw | wybutoxosine |
| p | pseudouridine |
| q | queosine |
| s2c | 2-thiocytidine |
| s2t | 5-methyl-2-thiouridine |
| s2u | 2-thiouridine |
| s4u | 4-thiouridine |
| t | 5-methyluridine |
| t6a | N-((9-β-D-ribofuranosylpurine-6-yl)carbamoyl)threoninetm |
| | 2′-O-methyl-5-methyluridine |
| um | 2′-O-methyluridine |
| yw | wybutosine |
| x | 3-(3-amino-3-carboxypropyl)uridine, (acp3)u |
| araU | β,D-arabinosyl |
| araT | β,D-arabinosyl |

According to a particularly preferred embodiment of the invention, alkaloid levels in latex of P.somniferum, particularly morphinan alkaloids, are modulated using sense or antisense sequences of a cyp80b1 gene which may be identical to the endogenous P.somniferum cyp80b1 gene or alternatively may be different from the endogenous P.somniferum cyp80b1 gene. In accordance with this embodiment a P.somniferum plant may therefore be engineered to express a cyp80b1 gene originating at least partially from a different plant species such as E. californica or C japonica.

Preferred variants for increasing alkaloid production in P.somniferum involve the use of a sequence comprising or consisting of a “sense” sequence encoding the P.somniferum CYP80B1 protein illustrated in FIG. 6 (SEQ. ID. N^{o} 1) or in FIG. 7 (SEQ. ID. N^{o} 3) as exogenous nucleic acid of the invention

Preferred variants for decreasing alkaloid production in P.somniferum involve the use of a sequence comprising or consisting of an “antisense” sequence encoding the P.somniferum CYP80B1 protein illustrated in FIG. 6 (SEQ. ID. N^{o} 1) or in FIG. 7 (SEQ. ID. N^{o} 3) as exogenous nucleic acid of the invention

According to the invention, the exogenous nucleic acids are introduced into plant cells using transfection or transformation techniques conventional in the art, in conditions allowing expression of the exogenous nucleic acids. A number of transformation techniques have been reported for Papaver. For example, microprojectile bombardment of cell suspension cultures may be used. Transformation may also be effected using Agrobacterium, particularly Agrobacterium tumefaciens (see for example WO 99/34663), or Agrobacterium rhizogenes, using either cell suspension cultures or tissue explants. A number of further techniques are available and are known to the skilled man.

With regard to regeneration of transgenic plants, techniques for recovering whole P. somniferum plants from tissue cultures are now known. Opium poppy has been regenerated via somatic embryogenesis (Nessler, 1982; Schuchmann and Wellmann, 1983; Yoshikawa and Furuya, 1983; Wakhlu and Bajwa, 1986; Ovecka et al., 1996; Kassem and Jacquin, 2001; Chitty et al., 2003), via anther culture and microspore-derived calluses and plants (Dieu et al., 1988), and via shoot organogenesis (Park and Facchini, 2000 a).

The invention also relates to a method for producing a plant having altered alkaloid biosynthesis, comprising
i) introducing into cells of a plant, an expressible exogenous nucleic acid comprising or consisting of an (S)-N-methylcoclaurine 3′-hydroxylase gene, or a derivative thereof, and
ii) optionally propagating the plant.

In the context of the present invention, the term “plant” refers to whole, differentiated higher plants. Plants are distinct from undifferentiated callus cell lines.

The propagation of the plant may be effected by means of self-fertilisation or cross-fertilisation, followed by selection of progeny plants having the capacity to exhibit altered alkaloid biosynthesis. Such plants may exhibit a phenotypically altered alkaloid production, or alternatively may only exhibit such a phenotypic trait on further propagation, depending upon whether the plant is homozygous or heterozygous for the change, and on whether the trait is dominant or recessive.

The propagation of the plant may also be effected by means of clonal propagation.

The invention also relates to methods for producing alkaloids comprising
producing a plant having altered alkaloid biosynthesis in accordance with any of the methods described herein,
harvesting the plant parts containing the alkaloids,
extracting the alkaloids from the plant parts.

The plant parts which are harvested are usually poppy capsules or straw or latex, although some species for example P.bracteatum contains alkaloids in the roots. Extraction is carried out in accordance with conventional techniques.

The invention further relates to the transgenic plants, particularly P.somniferum having altered alkaloid biosynthesis which can be obtained by the methods of the invention, and to progeny and seeds of such plants. The progeny plants carry the genetic alteration responsible for altered alkaloid biosynthesis, and may be homozygous or heterozygous for the trait. If a progeny plant is heterozygous for the desired characteristic, its phenotype will not necessarily show altered alkaloid biosynthesis, but such a plant can be used to introduce the trait into other lines. The plants of the invention contain in their genetic material, an exogenous nucleic acid comprising or consisting of an (S)-N-methylcoclaurine 3′-hydroxylase gene, or a derivative thereof. The plants can be distinguished from naturally occurring plants in that they either contain the (S)-N-methylcoclaurine 3′-hydroxylase gene at a copy number which is higher than that occurring naturally, or they contain an (S)-N-methylcoclaurine 3′-hydroxylase gene in a genetic location which is different from that occurring naturally, or the exogenous nucleic acid is heterologous with respect to the genome of the plant. “Heterologous” in this context means that the (S)-N-methylcoclaurine 3′-hydroxylase gene is not identical to the naturally occurring gene, for example it comprises heterologous transcription regulatory signals, or differences in nucleic acid sequence with respect to the endogenous gene. The regulatory signals used in the gene preferable permit expression of the exogenous nucleic acid in the vascular bundle of the plant.

Depending on the method used for introducing the exogenic nucleic acid, it may be stably integrated into the genome of the plant, or alternatively may be on an autonomously replicating virus or plasmid.

The invention also relates to plant cells, for example cell cultures or cell lines, containing in their genetic material, an exogenous nucleic acid comprising or consisting of an (S)-N-methylcoclaurine 3′-hydroxylase gene, or a derivative thereof.

The invention also relates to a nucleic acid encoding a (S)-N-methylcoclaurine 3′-hydroxylase, having the sequence illustrated in FIG. 7 (SEQ. ID. N^{o} 2), said nucleic acid preferably comprising the P.somniferum cyp80b1 coding sequence illustrated in FIG. 6 (SEQ. ID. N^{o} 1).

The invention also relates to a hybrid nucleic acid encoding a (S)-N-methylcoclaurine 3′-hydroxylase comprising a portion of the P.somniferum cyp80b1 coding sequence illustrated in FIG. 6 (SEQ. ID. N^{o} 1) and a portion of the E.californica cyp80b1 coding sequence illustrated in FIG. 11 (SEQ. ID. N^{o} 7) or FIG. 13 (SEQ. ID. N^{o} 9).

The invention also relates to a nucleic acid comprising or consisting of the 5′ regulatory sequences of the P.somniferum cyp80b1 gene illustrated in FIG. 9 (SEQ. ID. N^{o} 5). This sequence, or a functional part thereof, comprising or consisting of for example from around 2500 to around 3500 consecutive bases upstream of the ATG start codon in the sequence illustrated in FIG. 9, can be used in chimeric genes for initiation and/or control of transcription of genes in plants. It is particularly advantageous to use this promoter sequence for expression of heterologous genes in P.somniferum and other plants of the Papver genus.

The cDNA encoding the cytochrome P450-dependent enzyme (S)-N-methylcoclaurine 3′-hydroxylase of P. somniferum has been deposited with the DSMZ (Deutsche Sammiung von Mikroorganismen und Zellkulturen GmbH) on 14 Jul. 2005, under accession number DSMZ 17451. The deposited cDNA comprises a 1511 bp insert in pUC18, corresponding to the sequence illustrated in FIG. 8 (SEQ. ID. N^{o} 4) and encoding all but the first 6 amino acids of the P.somniferum (S)-N-methylcoclaurine 3′-hydroxylase (Huang & Kutchan, 2000). The deposited cDNA is designated cyp80b3 in accordance with recently proposed nomenclature, but is identical to the sequence illustrated in FIG. 8 (SEQ. ID. N^{o} 4) and formerly designated P.somniferum cyp80b1.

Further aspects of the invention are illustrated in the Figures.

### FIGURE LEGENDS

FIG. 1. Schematic presentation of the biosynthetic pathway from (S)-norcoclaurine to codeine, laudanine and (S)-scoulerine in P. somniferum. The position of the cDNA cyp80b1 used in this work is indicated in large print.

FIG. 2. Polymerase chain reaction amplification of transgene from genomic DNA from wild type (WT) and transgenic plants of the T₃ generation. Reaction mixtures were resolved by agarose gel electrophoresis and DNA was visualized with ethidium bromide. Lane 1) 1 kb+ molecular size marker; 2) negative control (water instead of DNA template); 3) negative control (genomic DNA from WT); 4) genomic DNA template from T₃-15.7/2; 5) genomic DNA template from T₃-15.7/13; 6) genomic DNA template from T₃-15.7/14; 7) genomic DNA template from T₃-17.6/10; 8) sense-cyp80b1 pPLEX X002; 9) antisense-cyp80b1 pPLEX X002; 10) 1 kb+ molecular size marker; 11) negative control (water instead of DNA template); 12) negative control (genomic DNA from WT); 13) genomic DNA template from T₃-15.7/2; 14) genomic DNA template from T₃-15.7/13; 15) genomic DNA template from T₃-15.7/14; 16) genomic DNA template from T₃-17.6/10; 17) sense-cyp80b1 pPLEX X002; 18) antisense-cyp80b1 pPLEX X002; 19) 1 kb+ molecular size marker. Lanes 2-9 are results from an amplification of the cyp80b1 transgene using oligodeoxynucleotide primers S4S4 and Me1′; lanes 11-18 are results from an amplification of a fragment of the nptll gene using oligodeoxynucleotide primers NPTIls and NPTIlas. The primer sequences are given in the Material and Methods.

FIG. 3. Comparative alkaloid analysis of latex from P. somniferum wild type (WT), a T₁ generation plant (T1-1850) that resulted from transformation with sense-cyp80b1 and a T₃ generation plant (T3-15.7/13) that resulted from transformation with sense-cyp80b1. WT values represent the average alkaloid content of 29 wild type plants. Alkaloids in latex were calculated as μg alkaloid/100 μg soluble protein, then normalized to 100% to produce the individual divisions within pie graphs, representing relative ratios of the latex alkaloids. Total WT alkaloids were set to 100% to produce the relative diameters of the pie graphs, representing the relative total alkaloid contents of the transgenic compared to the WT.

FIG. 4. Comparative alkaloid analysis of latex from P. somniferum wild type (WT) and two T₃ generation plants (T3-15.7/2 and T3-15.7/14) that resulted from transformation with sense-cyp80b1. WT values represent the average alkaloid content of 29 wild type plants. Alkaloids in latex were calculated as μg alkaloid/100 μg soluble protein, then normalized to 100% to produce the individual divisions within pie graphs, representing relative ratios of the latex alkaloids. Total WT alkaloids were set to 100% to produce the relative diameters of the pie graphs, representing the relative total alkaloid contents of the transgenic compared to the WT.

FIG. 5. Comparative alkaloid analysis of latex from P. somniferum wild type (WT), a T₁ generation plant (T1-579) that resulted from transformation with antisense-cyp80b1 and a T₃ generation plant (T3-17.6/10) that resulted from transformation with antisense-cyp80b1. WT values represent the average alkaloid content of 29 wild type plants, representing relative ratios of the latex alkaloids. Alkaloids in latex were calculated as pg alkaloid/100 μg soluble protein, then normalized to 100% to produce the individual divisions within pie graphs. Total WT alkaloids were set to 100% to produce the relative diameters of the pie graphs, representing the relative total alkaloid contents of the transgenic compared to the WT.

FIG. 6. Papaver somniferum cyp80b1 coding sequence and amino acid sequence (full length). Start and stop codons are indicated. (SEQ. ID. N^{o} 1) (SEQ. ID. N^{o} 2).

FIG. 7. Papaver somniferum CYP80B1 partial amino acid sequence. The first five amino acids and methionine are missing (GenBank accession number AAF 61400). (SEQ. ID. N^{o} 3).

FIG. 8. Papaver somniferum cyp80b1 partial cDNA sequence, coding for the amino acid sequence of FIG. 7. The first 17 nucleotides within the coding sequence are missing. The TAA stop codon is underlined (GenBank accession number AF 134590). (SEQ. ID. N^{o} 4).

FIG. 9. Papaver somniferum cyp80b1 promoter sequence, together with a 5′ portion of the coding sequence. ATG start codon is indicated. The position of the PCR primer used for genome walking to generate the 5′non-coding region is also indicated. (SEQ. ID. N^{o} 5).

FIG. 10. Eschscholzia californica CYP80B1V1 partial amino acid sequence. The first five amino acids are missing (GenBank accession number AAC 39452) (SEQ. ID. N^{o} 6).

FIG. 11. Eschscholzia californica cyp80b1v1 partial cDNA sequence, coding for the amino acid sequence of FIG. 10 (GenBank accession number AF 014800) (SEQ. ID. N^{o} 7).

FIG. 12. Eschscholzia californica CYP80B1V2 complete amino acid sequence (GenBank accession number AAC 39453). (SEQ. ID. N^{o} 8).

FIG. 13. Eschscholzia californica cyp80b1v2 cDNA sequence, coding for the amino acid sequence of FIG. 12 (GenBank accession number AF014801) (SEQ. ID. N^{o} 9).

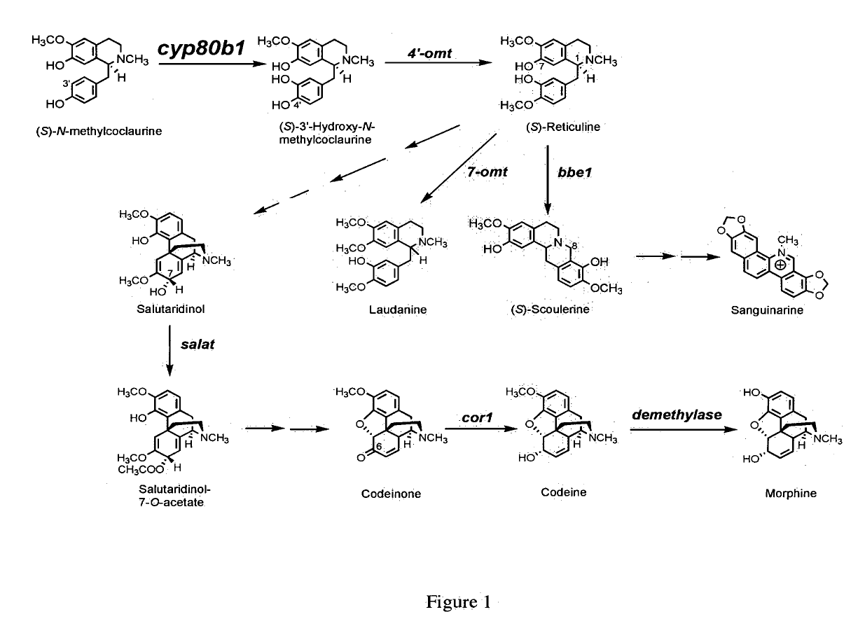

### EXAMPLES

The following examples describe the A. tumefaciens-mediated transformation and subsequent regeneration of opium poppy with introduction of an antisense- or sense-expressed (S)-N-methylcoclaurine 3′-hydroxylase encoding cDNA cyp80b1 into seedling explants. The transgene altered alkaloid levels in latex; sense-cyp80b1 expressing plants showed increased levels of alkaloids and antisense-cyp80b1 expressing plants showed reduced levels of alkaloids in latex.

### Example 1

### Material and Methods

1.1 Construction of Transformation Vectors

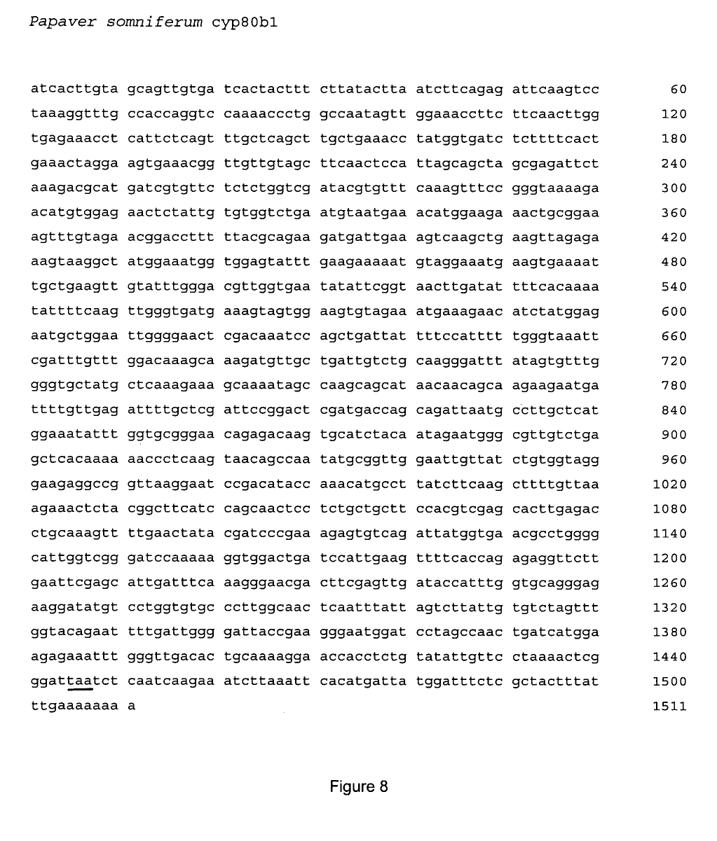

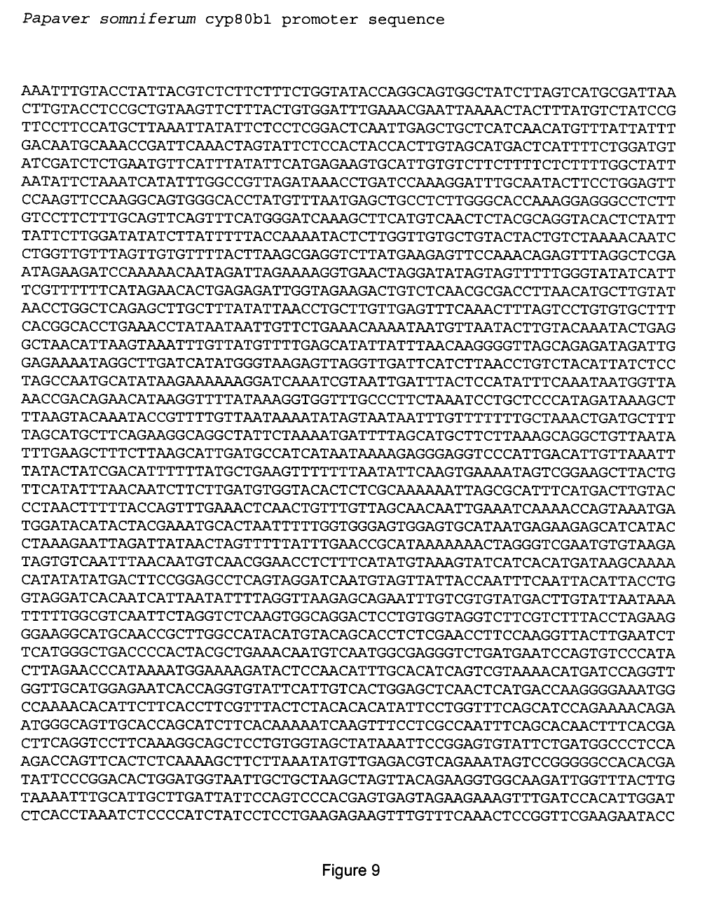

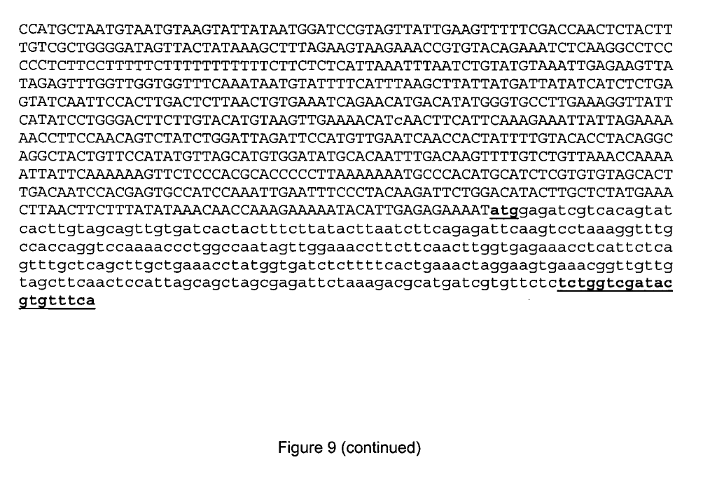

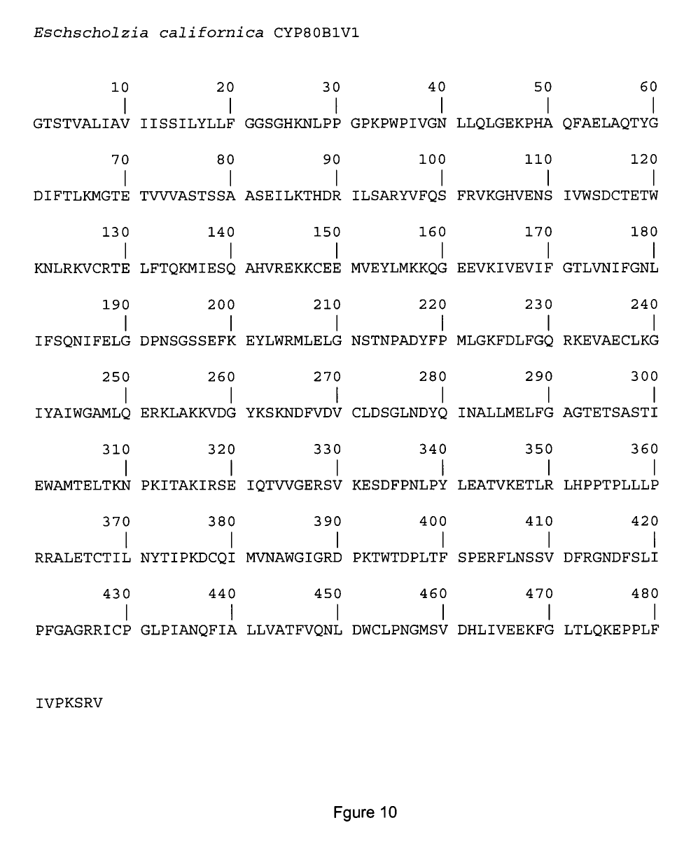

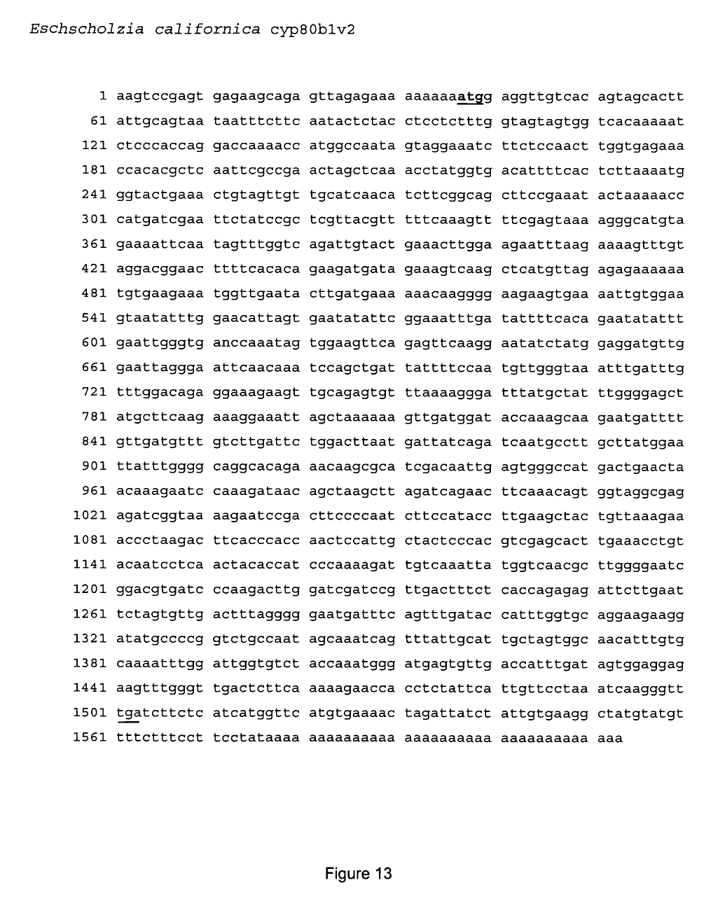

Recombinant cyp80b1 from P. somniferum L. was cloned into pUC-18 vector (Pauli and Kutchan, 1998; Huang and Kutchan, 2000). The cyp80b1 was a chimeric sequence composed of the partial P. somniferum coding sequence illustrated in FIG. 8 (SEQ. ID. N^{o} 4), (DSMZ 17451) completed at the 5′ end by nucleotides from the Eschscholzia califorica cyp80b1v2 sequence illustrated in FIG. 13 (SEQ. ID. N^{o} 9), encoding the first five amino terminal amino acids and methionine (ATG GAG GTT GTC ACA GTA). The BglII/XhoI restriction fragment was resolved by agarose gel electrophoresis, purified with the Wizard® PCR Preps DNA Purification System (Promega) and blunted with Pfu-polymerase for 30 min at 75° C. After a second purification with the Wizard® System, the blunted DNA fragment was ligated into the SnaBI digested vector pPLEX X002 (vector from Phil Larkin, CSIRO Plant Industry, Canberra).

The plasmids antisense-cyp80b1 and sense-cyp80b1 pPLEX X002 were transformed by heat shock (30 sec, 42° C.) in competent E. coli XL1-BlueMRF′. Positive transformants were plated onto Luria-Bertani medium (Maniatis et al., 1982) containing 100 mg/l ampicillin or 50 mg/l spectinomycin. Positive colonies growing on LB-spectinomycin but not on LB-ampicillin were used to inoculate an over night culture in LB medium containing 50 mg/l spectinomycin. The binary plasmids were isolated from 3 ml overnight cultures according to the method of Birnboim and Dolly (1979). The bacterial suspensions were centrifuged (5 min, 10000 rpm) and the pellets were resuspended in 200 μl GTE buffer (50 mM glucose, 25 mM Tris/HCl pH 8.0, 10 mM EDTA pH 8.0, 100 μg/ml RNAse A). The lysis of the cells occurred by adding 300 μl 0.2 M NaOH/1% (w/v) SDS to the solution.

After precipitation of proteins with 300 μl 3 M potassium acetate pH 4.8, the suspensions were incubated on ice for 10 min, centrifuged (10 min, 14000 rpm) and the clear supernatants were then transferred into new microcentrifuge tubes. Plasmid DNA was precipitated with 1 volume 100% isopropanol and pelleted by centrifugation (10 min, 14000 rpm). After washing with 500 μl 70% (v/v) ethanol, the pellet was dried 15 min at room temperature and resolved in 20 μl 0.1× TE buffer. The direction of the inserted cyp80b1 cDNA was confirmed by restriction enzyme digest with PacI/PvuI or PvuI prior to nucleotide sequence determination with ABI PRISM™ BigDye Terminator Cycle Sequencing Kit according to the manufacturer's instructions.

1.2 Preparation of A. tumefaciens

The binary vectors antisense-cyp80b1 and sense-cyp80b1 pPLEX X002 were transformed by electroporation (BioRad Gene Pulser, volts: 2,0 kV; resistance: 400 Ω; capacitance 25 μF) into the disarmed A. tumefaciens strain AGL1 (Lazo et al., 1991) and plated onto LB medium containing 50 mg/l spectinomycin, 20 mg/I rifampicin and 50 mg/l carbenicillin. Transformed A. tumefaciens cultures were grown to mid-log phase (A₆₀₀=0.5) at 28° C. on a gyratory shaker at 220 rpm in liquid LB medium containing 50 mg/l spectinomycin. The bacterial cells were collected by centrifugation for 10 min at 8000 rpm and resuspended at a cell density of A₆₀₀=0.3 in liquid LB medium. The solutions were used directly for the transformation of P. somniferum explants.

1.3 Seed Sterilization and Germination

The genotype of P. somniferum used in this study was the industrial inbreed line C048-6-14-64 obtained from Tasmanian Alkaloids Pty Ltd, Westbury. Seeds were surface-sterilized by washing for 1 min with 70% (v/v) ethanol and then in 0.8% (v/v) sodium hypochlorite solution plus 1-2 drops of autoclaved Triton-X-100 for 25 min with agitation, then rinsed five times in sterile distilled water or until the hypochlorite sent is removed. Seeds were blotted dry on sterile filter paper and 37 seeds were placed in 100×20 mm Petri dishes containing B5O medium, which consists of B5 macronutrients, micronutrients, iron salts and vitamins (Gamborg et al., 1968), 20 g/l sucrose, 2 g/l MES using 0.8% (w/v) Sigma agar as the gelling agent (Larkin et al., 1999; Chitty et al., 2003). pH was adjusted with 1M NaOH to 5.6 (Larkin et al., 1999; Chitty et al., 2003) and then sterilized by autoclaving at 121° C. for 20 min. Dishes were sealed with Micropore™ surgical tape (3M Pharmaceuticals) and stored at 4° C. for 24 h. Seeds were germinated in a growth chamber at 23° C. under standard daylight (OSRAM L36 W/72-965 BIOLUX, München, Germany) with a flux rate of 160 μMol/m²s, 0.5 m/s air circulation and a 16-h photoperiod.

1.4 Transformation, Selection and Regeneration of Transgenic Plants

Transgenic plants have been regenerated according to Larkin et al. (1999) and Chitty et al. (2003). Hypocotyls from 8-day-old seedlings were isolated by removing the root and the cotyledons bellow the cotyledonary node. The explants were immediately inoculated by immersion in a liquid A. tumefaciens culture for 15 min. Control explants were incubated in sterile distilled water. The hypocotyls were transferred directly to 19D medium. Callusing medium (CM) also referred to as 19D is identical to B5O except that it includes 1 mg/l 2,4-D (2,4-dichlorophenoxy acetic acid) (Larkin et al., 1999; Chitty et al., 2003). After four to five days of co-cultivation, the explants were washed in sterile, distilled water until the water was clear of Agrobacterium, blotted dry on sterile filter paper and transferred directly to 19D medium containing 150 mg/l timentin (GlaxoSmithKline, Worthing; Duchefa Biochemie BV, Haarlem) and 25 mg/l paromomycin. Control explants were incubated on 19D medium. Four months after transformation, embryogenic callus referred to as type 11 (Larkin et al, 1999; Chitty et al., 2003) developed and was transferred to B5O medium lacking growth regulators containing 150 mg/l timentin and 25 mg/l paromomycin. Control calli were incubated on B5O medium without antibiotics. All cultures were transferred after 21 days to fresh medium and were grown in a growth chamber at 23° C. under standard daylight as described above. After eight weeks on B5O medium, fully differentiated somatic embryos developed and were grown to plantlets in tissue culture, then after sufficient shoot and root growth, the plants were transferred to soil (80% compost, 20% perlite). The transgenic as well as control plants were grown in a green house under a High Pressure Sodium Lighting System (Philips, SON-T AGRO 400W) at 20-24° C. with a 18-20° C. temperature shift at night, a relative humidity of 60% and a 16-h photoperiod. The opium poppy plants regenerated directly from a callus were designated as the T₀ generation and their self-pollinated progeny as the T₁, T₂ and T₃generations.

1.5 Harvest of Leaves, Roots and Latex

When the plants reached a size of at least 20 cm, leaves were collected and used for the isolation of DNA and RNA. The poppy flower was self-pollinated on the day the petals opened. Exactly two days after the petals dropped, latex was collected by incising the capsule longitudinally with a scalpel. The exuded latex was collected with a pipette and was stored in 200 μl collection buffer (100 mM potassium phosphate pH 7.2, 500 mM D-mannitol, 20 mM ascorbic acid) (Decker et al., 2000) and frozen in liquid nitrogen. The samples were stored at −80° C. prior to analysis. After ripening of the capsule, the seeds were collected and after determination of root weight, the roots were frozen in liquid nitrogen and were stored at −80° C. prior to analysis.

1.6 Nucleic Acid Isolation

Plant genomic DNA and total RNA was extracted with modified standard protocols as described in Maniatis et al. (1982) and Cathala et al. (1983). All steps if not otherwise mentioned were carried out at room temperature. 1 g leaf material was ground in a mortar with liquid nitrogen. The tissue was mixed with 3.5 ml lysis buffer (10 mM Tris/HCl pH 7.5, 50 mM NaCl, 1% [w/v] SDS, 4% [w/v] PVPP, 1 mM EDTA pH 8.0, 14 mM β-mercaptoethanol) and 3.5 ml phenol-chloroform-isoamylalcohol (25:24:1) and extracted for 30 min on a gyratory shaker. After 10 min centrifugation (5000 rpm) the aqueous layer was first extracted with 3.5 ml phenol-chloroform-isoamylalcohol and after a centrifugation as described above extracted with 3 ml chloroform. The phases were separated by centrifugation (10 min, 5000 rpm) and the aqueous layer was then mixed with 0.1 volume 3 M sodium acetate pH 5.2, 1 volume isopropanol and incubated at −20° C. for 1 h. After centrifugation (20 min, 5000 rpm) the pellet was washed with 70% (v/v) ethanol, dried at 37° C. and was resuspended in 300 μl TE (7.5 mM Tris/HCl pH 8.0, 0.75 mM EDTA) buffer. The total RNA was precipitated over night at 4° C. with 300 μl 6 M LiCl. On the next morning the solution was centrifuged for 15 min with 14000 rpm at 4° C. The RNA pellet was washed with 500 μl 70% (v/v) ethanol, dried at 37° C. and resuspended in 50 μl TE buffer. The DNA containing supernatant was precipitated with 0.1 volume 3 M sodium acetate pH 5.2, 1 volume isopropanol and incubated at −20° C. for 20 min. After centrifugation (15 min, 14000 rpm, 4° C.) the pellet was washed with 500 μl 70% (v/v) ethanol, dried at 37° C. and resuspended in 50 μl TE buffer.

1.7 Polymerase Chain Reaction Analysis of Transformation

PCR was used to test the transformation of transgenic plants. DNA amplification was performed under the following conditions: a) for nptll: Primer sequences, NPTIls: 5′-GAG GCT ATT CGG CTA TGA CTG-3′and NPTIlas: 5′-ATC GGG AGC GGC GAT ACC GTA-3′ (Bonhomme et al., 2000); cycle , 94° C., 3 min, 35 cycles of 94° C., 30 sec; 60° C., 30 sec; 72° C., 1 min, cycle 72° C., 5 min. At the end of all cycles the reaction was cooled to 4° C.; b) for cyp80b1 (S4S4 promoter and Me1′ terminator): Primer sequences, S4S4: 5′-TM GCG TAC TCA GTA CGC TTC-3′ and Me1′: 5′-GCA TTA CAA CAT GCA TCT GAC-3′; cycle, 94° C., 3 min, 35 cycles of 94° C., 1 min; 55° C., 1 min; 72° C., 2 min, cycle 72° C., 5 min. At the end of all cycles, the reaction was cooled to 4° C. The amplified DNA was resolved by agarose gel electrophoresis using standard protocols.

1.8 HPLC Analysis of Benzylisoquinoline Alkaloids

Latex samples of wild type or transgenic plants were thawed on ice and treated 6 min in a ultrasound waterbath at 4° C. The sample was centrifuged 30 min with 12500 rpm at 4° C. The supernatant was used to determine the protein concentration. An aliquot of the supernatant was mixed with an internal standard (dihydrocodeinone), diluted with 70% (v/v) ethanol and centrifuged. The pellet, to which an internal standard was also added, was treated under sonification with a defined volume of 70% (v/v) ethanol and was separated on a reversed phase column. Samples were analysed by HPLC on a liquid chromatography system (Agilent Technologies, Waldbronn, Germany) using a reverse phase column (LiChrospher 60 RP-select B, 4×250 mm, 5 μm, UV detection at 210, 282 and 440 nm). The alkaloids were separated at a flow rate 1 ml min⁻¹ and using the gradient H₂O: acetonitrile (0-25 min: 0-46% acetonitrile, 25-26 min 46-100% acetonitrile, 26-33 min 100% acetonitrile) containing 0.01% (v/v) phosphoric acid. Peaks have been routinely identified from their UV spectra and by comparison of their retention times to those of authentic standards. Subsequently the identity of the peaks was confirmed by liquid chromatography-mass spectroscopy.

Root samples were homogenized with 10 ml 70% (v/v) ethanol and 20 μg dihydrocodeinone as internal standard. The filtrate was evaporated, made basic with sodium bicarbonate and subsequently extracted with ethyl acetate. The concentrated samples were dissolved in 500 μl 70% (v/v) ethanol and an aliquot was analysed by HPLC using the equipment as described above with a modified gradient as follows: 0-25 min: 0-60% acetonitrile, 25-26 min 100% acetonitrile, 26-33 min 100% acetonitrile and UV detection at 282 nm.

1.9 Liquid Chromatography-Mass Spectroscopy

The positive electrospray ionisation (ESI) mass spectra were obtained with a Mariner-TOF 5232 instrument (Applied Biosystems, Weiterstadt, Germany) using a turbulon spray (spray tip potential 5500V, SCIEX heater 310° C., nozzle potential 160 V, nebulizer and auxiliary gas (nitrogen) coupled to a modified Agilent 1100-LC equipped with a Lichrospher 60 RP-select B column (2×125 mm i.d.; 5 μm)). The analysis was performed with a gradient system consisting of H₂O—CH₃CN (each containing 0.2% HCOOH) as described for the HPLC analysis of root samples. Retention times and (M+H)⁺ ions were as follows: morphine (7.8 min; m/z 286.1); dihydrocodeine (10.7 min; m/z 302.1); codeine (11.0 min; m/z 300.1); oripavine (12.4 min; m/z 298.1); reticuline (14.1 min; m/z 330.1); scoulerine, 1,2-dehydroreticuline, salutaridine (14.8 min; m/z 328.1); laudanine (15.7 min; m/z 344.1); thebaine (17.0 min; m/z 312.1) laudanosine (17.5 min; m/z 358.1).

The MS-spectra of the alkaloids salutaridine, 1,2-dehydroreticuline and scoulerine were characterized by the molecular ion peak at m/z 328.1. To distinguish between these three compounds the selected reaction monitoring (SRM) mode was applied. The positive ion electrospray (ES) selected reaction monitoring data were obtained from a Finnigan MAT TSQ 7000 instrument (electrospray voltage 4.5 V; APIClD offset voltage 10V, heated capillary temperature 220° C.; sheath gas: nitrogen) coupled with a Surveyor MicroLC system (Finnigan) equipped with a RP-18 column (4 μm, 1×100 mm Ultrasep). For the HPLC a gradient system was used starting from H₂O: CH₃CN 85:15 (each of them containing 0.2% HOAc) to 5:95 within 30 min followed by a 10 min isocratic period; flow rate 70 ∞l min⁻¹. The following reactions based on the most prominent ion in the corresponding electrospray. CIDMS were used both for identification and quantification. A brief description of the SRM-method is given by Niessen (1999).

Full scan CIDMS data of the alkaloids (Raith et al., 2003):

dehydroreticuline: positive ion electrospray CIDMS, m/z (rel. int. %): 328 ([M]⁺, 11), 313 (13), 312 ([M-CH₃—H]⁺, 100), 296 (6), 284 (41), 267 (7), 204 (5), 190 (10), 176 (8).

salutaridine: positive ion electrospray CIDMS, m/z (rel. int. %): 328 ([M+H]⁺, 37), 297 (11), 285 (23), 282 (26), 270 (34), 265 (35), 255 (22), 239 (41), 237 (100), 233 (18), 211 (45), 207 (39), 205 (18), 183 (15), 58 (50), 44 (14).

scoulerine: positive ion electrospray CIDMS, m/z (rel. int. %): 328 ([M+H]⁺, 40), 296 (3), 178 (100), 176 (3), 151 (15).

### Example 2

### Results and Discussion

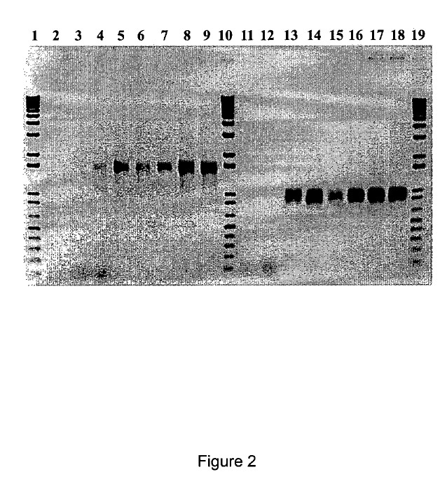

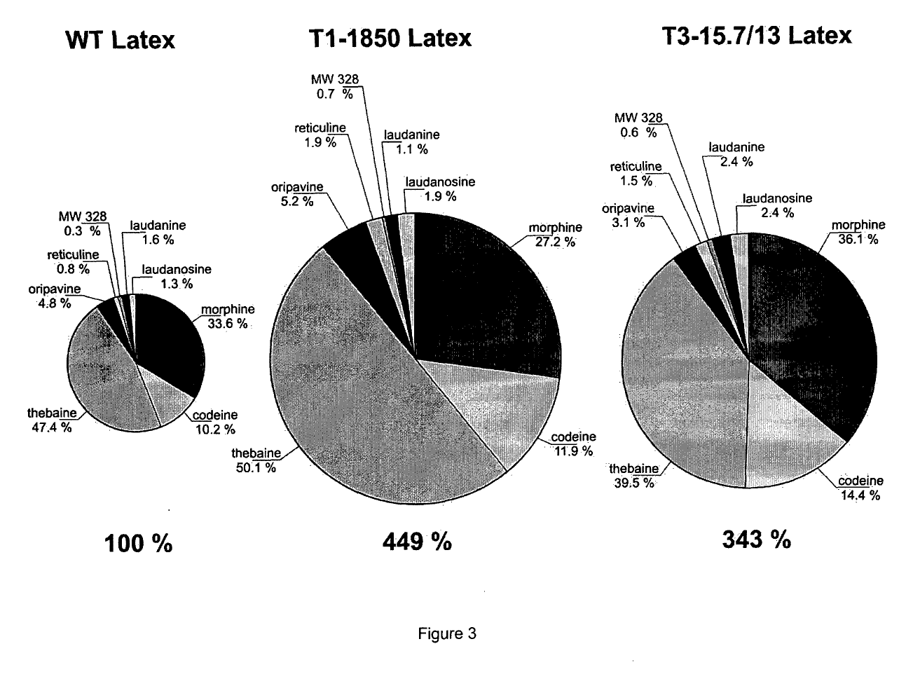

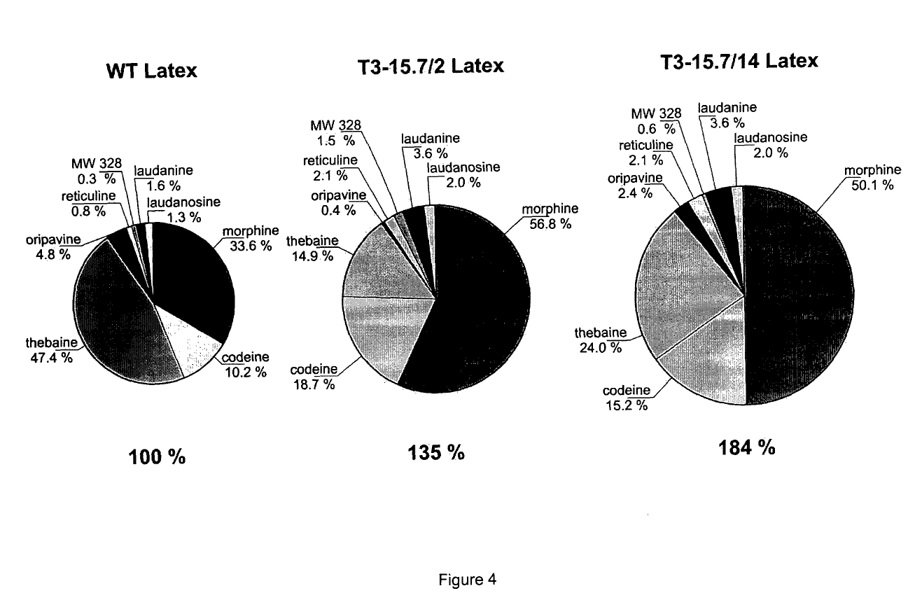

The presence of the transgene in regenerated P. somniferum plants was confirmed by the polymerase chain reaction (PCR) using genomic DNA as template and the appropriate oligodeoxynucleotide primers as given in the Material and Methods. Both the alkaloid biosynthetic gene cyp80b1 encoding (S)-N-methylcoclaurine 3′-hydroxylase and the kanamycin resistance gene nptll encoding neomycin phosphotransferase type 11 were detected in genomic DNA isolated from T₃ generation transgenic plants (FIG. 2). Introduction of the sense-cyp80b1 cDNA into P. somniferum by Agrobacterium-mediated transformation resulted in an overall increase of total alkaloids that was detectable in the T₁ generation and stable through at least the T₃ generation (FIG. 3). In some cases, as shown in FIG. 3, the relative ratios of the individual alkaloids remained constant, in others, such as shown in FIG. 4, this ratio was altered. Specific to the transgenic plants shown in FIG. 4, the relative amount of morphine was increased with respect to the other alkaloids, and the total alkaloid content was increased as well.

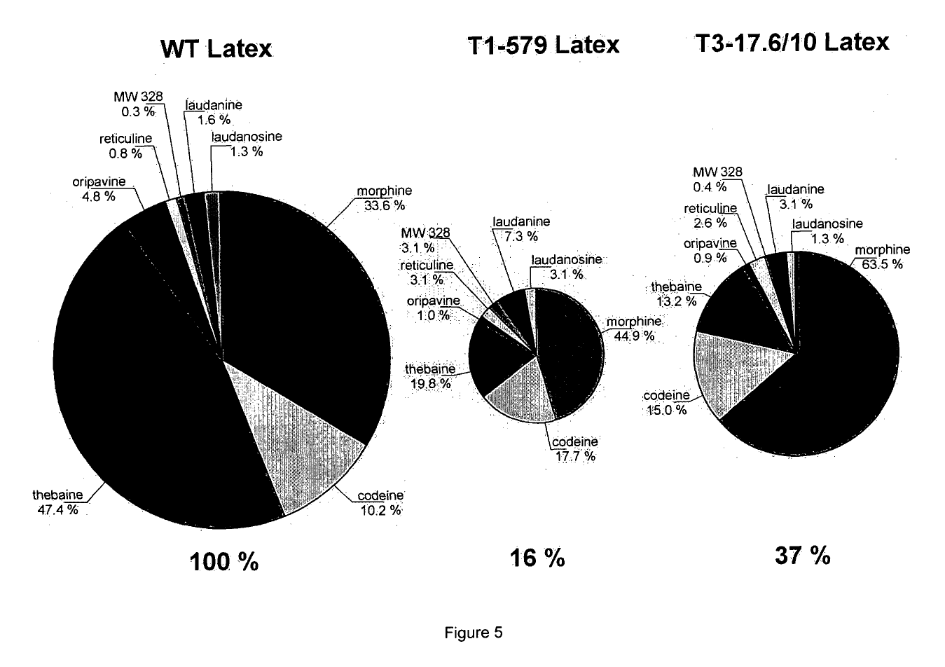

An important control in this type of metabolic engineering experiment is to demonstrate that the altered alkaloid profile is related to the presence of the transgene. To address this question, P. somniferum was transformed with the antisense-cyp80b1 cDNA. This resulted in an overall decrease in the amount of alkaloid present in the T₁ generation and this phenotype was stable through at least the T₃ generation (FIG. 5). In the example shown in FIG. 5, the ratio of alkaloids was also altered.

In summary, the cytochrome P-450-dependent monooxygenase CYP80B1 that lies on the biosynthetic pathway to the benzylisoquinoline alkaloid branchpoint intermediate (S)-reticuline appears to limit the ability of P. somniferum to synthesize alkaloid. Overexpression of the cDNA cyp80b1 in this plant resulted in an up to four-fold increase in the amount of total alkaloid in latex. This increase occurred either without changing the ratio of the individual alkaloids, or together with an overall increase in the ratio of morphine. Correspondingly, antisense-cyp80b1 expressed in P. somniferum resulted in a reduction of total alkaloid in latex, suggesting that the observed phenotypes were dependent on the presence of the transgene. Transformation with cyp80b1 can thus be used to modulate the alkaloid productivity of P. somniferum narcotic and condiment/oilseed cultivars.

### REFERENCES

Birnboim H C and Dolly J (1979) A rapid alkaline extraction procedure for screening recombinant plasmid DNA. Nucl. Acids Res. 7, 1513-1523
Brownstein M J (1993) A brief history of opiates, opioid peptides and opioid receptors. Proc. Natl. Acad. Sci. 90, 5391-5393
Cathala G, Savouret J-F, Mendez B, West B L, Karin M, Martial J A, and Baxter J D (1983) A method for isolation of intact translationally active ribonucleic acid. DNA 2 (4), 329-335
Chitty J A, Allen R S, Fist A J and Larkin P J (2003) Genetic transformation in commercial Tasmanian cultivars of opium poppy, Papaver somniferum, and movement of transgenic pollen in the field. Functional Plant Biology 30 (10), 1045-1058
Decker G, Wanner G, Zenk M H and Lottspeich F (2000) Characterization of the proteins in latex of the opium poppy (Papaver somniferum) using two-dimensional gel electrophoresis and microsequencing. Electrophoresis 21, 3500-3516
Dieu P and Dunwell J M (1988) Anther culture with different genotypes of opium poppy (Papaver somniferum L.): effect of cold treatment. Plant Cell, Tissue and Organ Culture 12, 263-271
Gamborg O L, Miller R A and Ojima K (1968) Nutrient requirements of suspension cultures of soybean root cells. Exp. Cells Res. 50, 151-158
Huang F C and Kutchan T M (2000) Distribution of morphinan and benzo[c]phenanthridine alkaloid gene transcript accumulation in i Papaver somniferum. Phytochemistry 53, 555-564
Husain A and Sharma J R (eds.) (1983) The opium poppy. A Monograph. Central Institute of Medicinal and Aromatic Plants, Lucknow, India
Kassem A and Jacquin A (2001) Somatic embryogenesis, rhizogenesis and morphinan alkaloids production in two species of opium poppy (2001) Journal of Biomedicine and Biotechnology 1, 70-78
Kutchan T M (1998) Molecular genetics of plant alkaloid biosynthesis. In: The alkaloids Vol. 50, (ed. G. Cordell) Academic Press, San Diego, 253-316.
Larkin P J, Chitty J A and Brettell R I S (1999) Methods for plant transformation and regeneration. International patent publication number WO 99/34663
Lazo G R, Stein P A and Ludwig R A (1991) A DNA transformation-competent Arabidopsis genomic library in Agrobacterium. Bio/Technology 9, 963-967
Maniatis T, Fritsch E F, and Sambrook J (eds.) (1982) Molecular cloning: A laboratory manual. Cold Spring Harbor Laboratory Press, Cold Spring Harbor, N.Y.
Nessler C L (1982) Somatic embryogenesis in the opium poppy Papaver somniferum. Physiologia Plantarum 55, 453-458
Niessen W M A (1999) Liquid Chromatography-Mass Spectrometry, Chapter 2: In Introduction to Mass Spectrometry, 67-69, second edition, Marcel Decker 1999
Ovecka M, Bobak M, Erdelsky K, Samaj J, Blehova A and Kristin J (1996) Morphology and conversion ability of somatic embryos in long term embryogenic callus culture of Papaver somniferum. Biologia 51, 417-423
Pauli, H H and Kutchan, T M (1998) Molecular cloning and functional heterologous expression of two alleles encoding (S)-N-methylcoclaurine 3′-hydroxylase (CYP80B1), a new methyl jasmonate-inducible cytochrome P-450 dependent monooxygenase of benzylisoquinoline alkaloid biosynthesis. Plant J. 13, 793-801
Park S U and Facchini P (2000) Agrobacterium-mediated transformation of opium poppy, Papaver somniferum, via shoot organogenesis. J. Plant Physiol. 157, 207-214
Raith K, Neubert R, Poeaknapo C, Boettcher C, Zenk M H and Schmidt J (2003) Electrospray tandem mass spectrometric investigations of morphinans. J. Am. Soc. Mass Spectrom. 14, 1262-1269
Schuchmann R and Wellmann E (1983) Somatic embryogenesis of tissue cultures of Papaver somniferum und Papaver orientale and its relationship to alkaloid and lipid matabolism. Plant Cell Rep. 2, 88-91
Wakhlu A K and Bajwa P S (1986) Regeneration of uniform plants from somatic embryos of Papaver somniferum (opium poppy). Phytomorphology 361, 101-105
Ye K, Ke Y, Keshava N, Shanks J, Kapp J A, Tekmal R R, Petros J and Joshi H C (1998) Opium alkaloid noscapine is an antitumor agent that arrests metaphase and induces apoptosis in dividing cells. Proc. Natl. Acad. Sci. 95, 1601-1606
Yoshikawa T and Furuya T (1983) Regeneration and in vitro flowering of plants derived from callus cultures of opium poppy (Papaver somniferum). Experientia 39: 1031-1033

1. Method for altering alkaloid biosynthesis in a plant, said method comprising modifying the expression of an (S)-N-methylcoclaurine 3′-hydroxylase gene in the plant.

2. Method according to claim 1 wherein the modifying comprises induction, enhancement, suppression or inhibition of expression of endogenous (S)-N-methylcoclaurine 3′-hydroxylase gene sequences in the plant.

3. Method of claim 1 for altering alkaloid biosynthesis in a plant, comprising:
i) introducing into cells of a plant, an expressible exogenous nucleic acid comprising or consisting of an (S)-N-methylcoclaurine 3′-hydroxylase, or a derivative thereof, and
ii) optionally propagating the plant,
wherein expression of the exogenous nucleic acid in the plant or in its progeny results in altered levels of alkaloid biosynthesis.

4. Method according to claim 3 wherein expression of the exogenous nucleic acid gives rise to over-expression of (S)-N-methylcoclaurine 3′-hydroxylase in the plant.

5. Method according to claim 3 wherein expression of the exogenous nucleic acid gives rise to decreased or abolished expression of (S)-N-methylcoclaurine 3′-hydroxylase in the plant.

6. Method according to claim 1 wherein the plant is a plant producing endogenous benzylisoquinoline-derived alkaloids.

7. Method according to claim 6 wherein the plant is a plant belonging to the order Ranunculales.

8. Method according to claim 7 wherein the plant is a plant belonging to the family Papaveraceae, Euphorbiaceae, Berberidaceae, Fumariaceae or Ranunculaceae.

9. Method according to claim 8 wherein the plant belongs to the genus Papaver.

10. Method according to claim 9 wherein the plant belongs to a species selected from Papaver somniferum, Papaver bracteatum, Papaver setigerum, Papaver orientate, Papaver pseudo-orientale, Papaver cylindricum, or Papaver rhoeas.

11. Method according to claim 3, wherein the exogenous nucleic acid is a cyp80b1 gene of Papaver somniferum or a cyp80b1 gene of Eschscholzia californica.

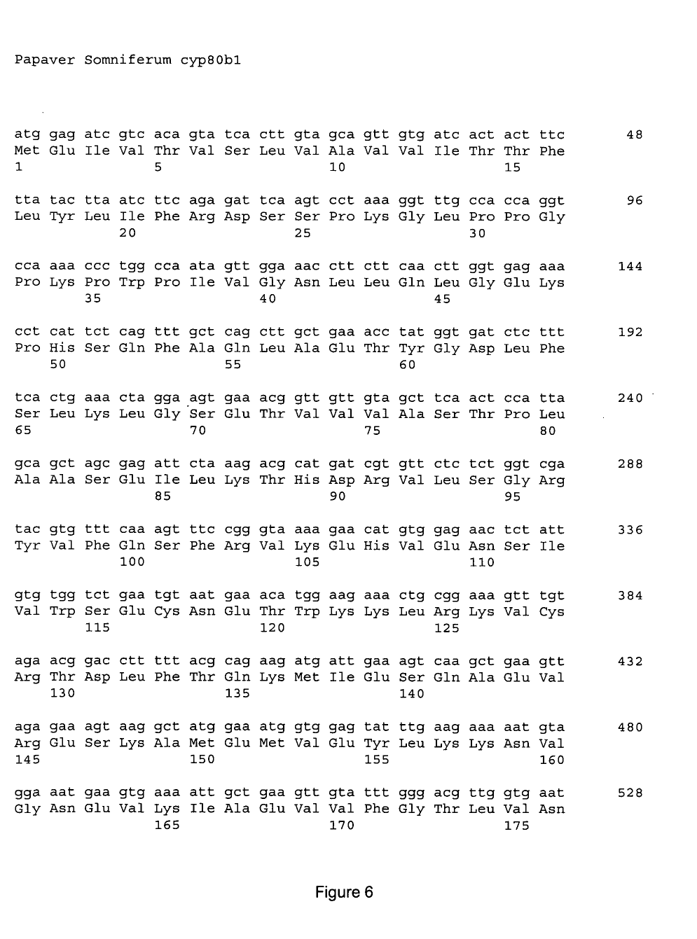

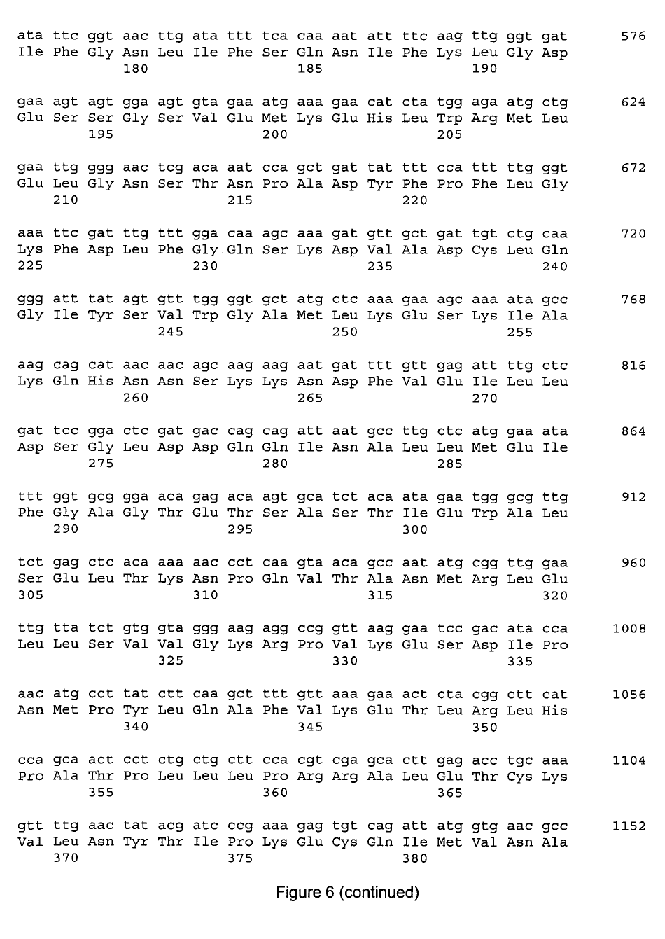

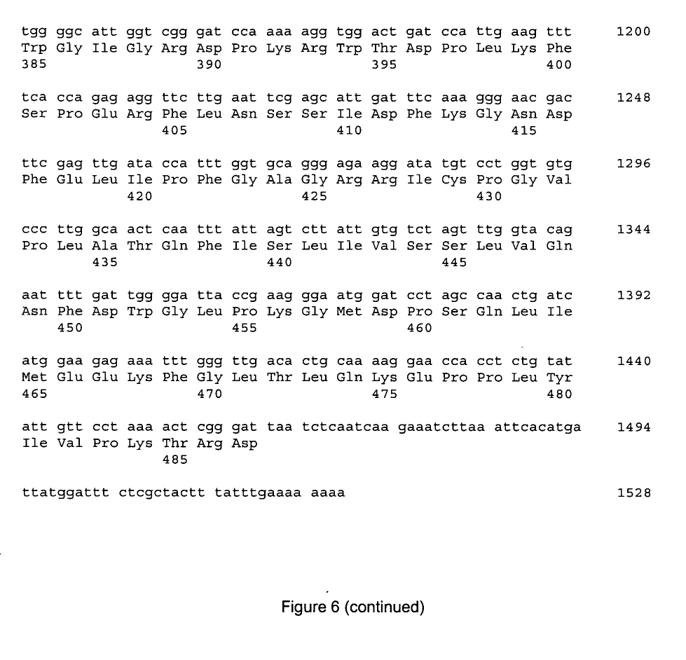

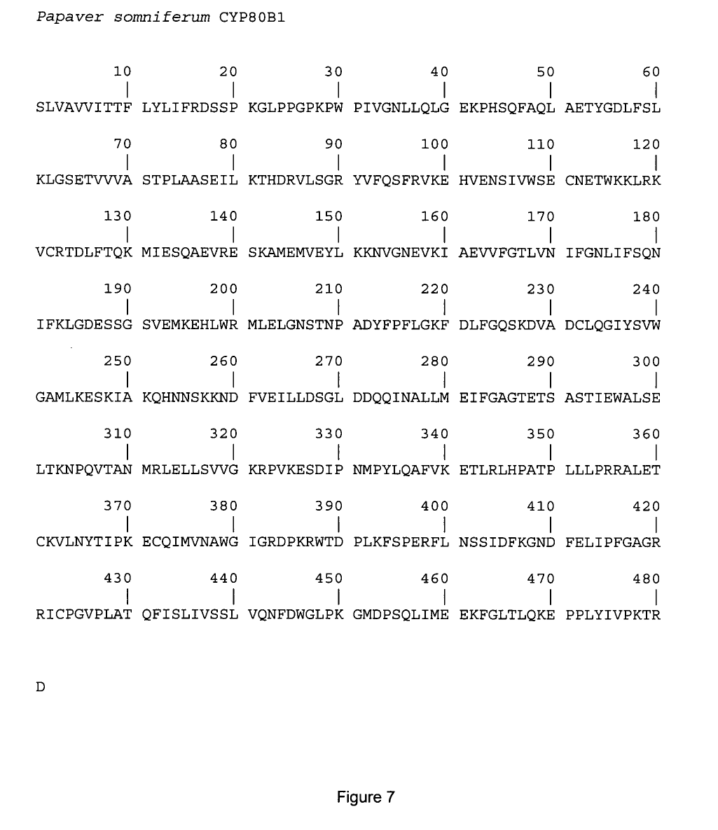

12. Method according to claim 11 wherein the cyp80b1 gene encodes a (S)-N-methylcoclaurine 3′-hydroxylase protein having the amino acid sequence illustrated in FIG. 6 (SEQ. ID. N^{o} 2) or the amino acid sequence illustrated in FIG. 7 (SEQ. ID. N^{o} 3).

13. Method according to claim 3 wherein the exogenous nucleic acid is a derivative of an (S)-N-methylcoclaurine 3′-hydroxylase gene, said derivative comprising a single or double stranded sequence variant, a fragment, a complementary sequence, an RNA equivalent, a mixed DNA/RNA equivalent, or an analogue of said gene.

14. Method according to claim 13, wherein the derivative is a derivative of a cyp80b1 gene of Papaver somniferum.

15. Method according to claim 14, wherein the derivative is chosen from:
i) a sequence encoding the protein sequence illustrated in FIG. 6 (SEQ. ID. N^{o} 2),
ii) a variant of the nucleotide sequence illustrated in FIG. 6 (SEQ. ID. N^{o} 2), said variant having at least 70% identity with the sequence of FIG. 6 (SEQ. ID. N^{o} 2) over a length of at least 1000 bases, and encoding a (S)-N-methylcoclaurine 3′-hydroxylase, or
iii) a fragment of sequence (i) or (ii), said fragment having a length of at least 20 nucleotides, or
iv) a sequence complementary to any one of sequences (i), (ii) or (iii), and having a length of at least 20 nucleotides, or
v) any one of sequences (i), (ii), (iii) or (iv) in double-stranded form, or
vi) the RNA equivalent of any of sequences (i), (ii), (iii), (iv) or (v).

16. Method according to claim 15, wherein the derivative is a variant of the coding sequence illustrated in FIG. 6 (SEQ. ID. N^{o} 1), wherein said variant has at least 85% identity with the sequence of FIG. 6 (SEQ. ID. N^{o} 1) over a length of at least 1000 bases, and differs from the sequence of FIG. 6 by insertion, replacement and/or deletion of at least one nucleotide.

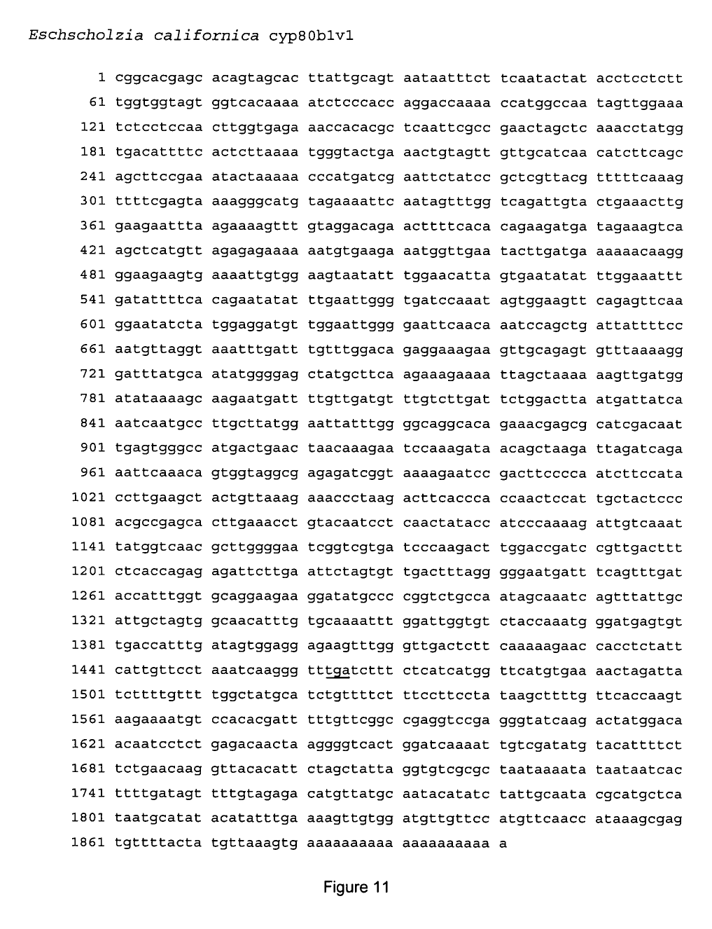

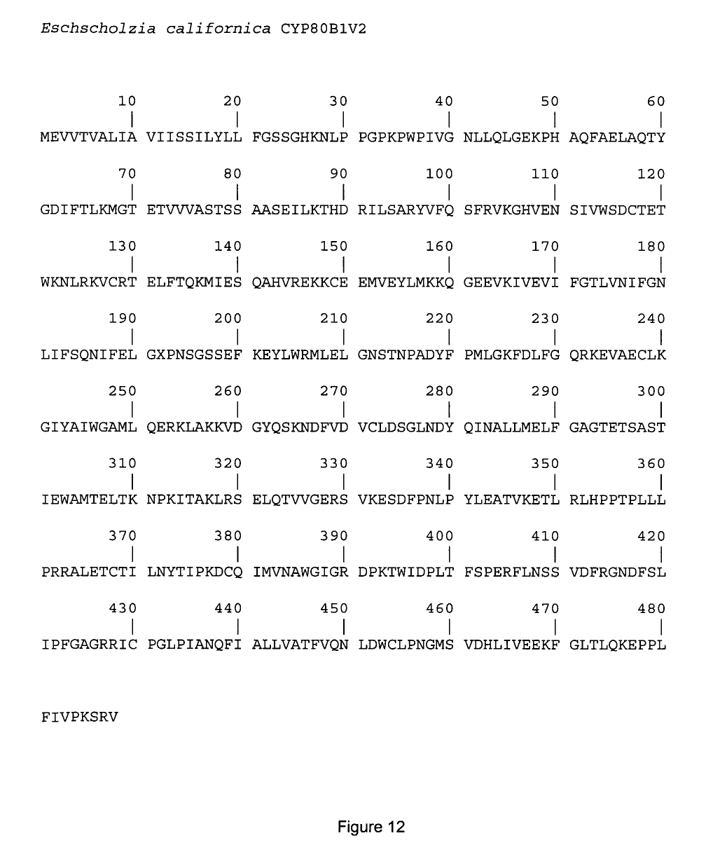

17. Method according to claim 16, wherein the derivative is a variant comprising a coding sequence as illustrated in FIG. 11 (SEQ. ID. N^{o} 7) or FIG. 13 (SEQ. ID. N^{o} 9) or a portion of said coding sequence.

18. Method according to claim 16, wherein the variant has the capacity to hybridise to the sequence illustrated in FIG. 6 (SEQ. ID. N^{o} 1), or its complement, in stringent conditions.

19. Method according to claim 18, wherein the variant has at least 99% identity with the nucleotide sequence of FIG. 6 (SEQ. ID. N^{o} 1) over a length of at least 1000 bases, and differs from the sequence of FIG. 6 (SEQ. ID. N^{o} 1) by insertion, replacement and/or deletion of at least one nucleotide.

20. Method according to claim 19, wherein the variant comprises an allelic variant of the cyp80b1 gene of Papaver somniferum illustrated in FIG. 7 (SEQ. ID. N^{o} 3), or a portion thereof.

21. Method according to claim 15, wherein the variant is a hybrid sequence comprising a portion of the cyp80b1 gene of P. somniferum and a portion of a cyp80b1 gene of a plant species other than P. somniferum.

22. Method according to claim 15, wherein the derivative consists of, or comprises a fragment of the nucleotide sequence illustrated in FIG. 6 (SEQ. ID. N^{o} 1), said fragment having a length of 25 to 1400 nucleotides.

23. Method according to claim 22, wherein the derivative consists of, or comprises a fragment of the nucleotide sequence illustrated in FIG. 6 (SEQ. ID. N^{o} 1), said fragment having a length of 60 to 500 nucleotides.

24. Method according to claim 15, wherein the derivative consists of, or comprises a sequence which is complementary to the nucleotide sequence illustrated in FIG. 6 (SEQ. ID. N^{o} 1) or to a part thereof, or to the sequence illustrated in FIG. 11 (SEQ. ID. N^{o} 7) or a part thereof, or to the sequence illustrated in FIG. 13 (SEQ. ID. N^{o} 9) or a part thereof.

25. Method according to claim 15, wherein the derivative comprises an antisense sequence, a ribozyme sequence, a DNAzyme sequence, an RNA-interference sequence, or a sequence capable of giving rise to an antisense sequence, a ribozyme sequence, a DNAzyme sequence, an RNA-interference sequence or the complement of an antisense sequence, a ribozyme sequence, a DNAzyme sequence, an RNA-interference sequence.

26. Method according to claim 15 wherein the derivative comprises at least one nucleotide analogue.

27. Method according to claim 1, wherein the derivative comprises a chimeric gene containing heterologous regulatory signals.

28. Method according to claim 1 wherein the altered alkaloid biosynthesis affects levels of alkaloids of the morphine and laudanine biosynthetic pathways.

29. Method according to claim 28, wherein the altered alkaloid biosynthesis affects levels of at least one alkaloid selected from the group consisting of morphine, codeine, codeinone, thebaine, oripavine, reticuline, (S)-laudanine and laudanosine.

30. Method according to claim 28 wherein the plant is a plant of the genus Papaver, and the altered alkaloid biosynthesis results in an increase in the amount of total alkaloid in latex of the plant.

31. Method according to claim 28 wherein the plant is a plant of the genus Papaver, and the altered alkaloid biosynthesis results in a decrease in the amount of total alkaloid in latex of the plant.

32. Method according to claim 28 wherein the plant is a plant of the genus Papaver and the altered alkaloid biosynthesis results in a change in the proportions of the individual alkaloids in the latex of the plant.

33. Method according to claim 32 wherein the altered alkaloid biosynthesis results in an increase in the proportion of the morphine levels in the latex of the plant.

34. Method according to claim 32 wherein the amount of total alkaloid in latex of the plant remains unchanged.

35. Method according to claim 3 wherein the plant is P. somniferum, and the exogenous nucleic acid comprises or consists of a sense sequence encoding the P.somniferum CYP80B1 protein illustrated in FIG. 6 (SEQ. ID. N″ 2) or a part thereof.

36. Method according to claim 3 wherein the plant is P.somniferum, and the exogenous nucleic acid comprises or consists of an antisense sequence of a sequence encoding the P. somniferum CYP80B1 protein illustrated in FIG. 6 (SEQ. ID. N^{o} 2) or a part thereof.

37. Method according to claim 3 wherein the exogenous nucleic acid is introduced into the plant genome by means of transformation with Agrobacterium.

38. Method according to claim 37 wherein the Agrobacterium is Agrobacterium tumefaciens.

39. Method for producing a plant having altered alkaloid biosynthesis, comprising
i) modifying the expression of an (S)-N-methylcoclaurine 3′-hydroxylase gene in the plant by a method according to claim 1, and
ii) optionally propagating the plant.

40. Method according to claim 39 wherein the propagation of the plant is effected by means of self-fertilisation or cross-fertilisation, followed by selection of progeny plants having the capacity to exhibit altered alkaloid biosynthesis.

41. Method according to claim 39 wherein the propagation of the plant is effected by means of clonal propagation.

42. Method according to claim 39 wherein the plant is P.somniferum.

43. Method for producing alkaloids comprising:
i) producing a plant having altered alkaloid biosynthesis, by the method of claim 39,
ii) harvesting the plant parts containing the alkaloids,
iii) extracting the alkaloids from the plant parts.

44. Method according to claim 43, wherein the plant is P.somniferum and the plant parts which are harvested are poppy capsules or straw or latex, and the alkaloids extracted are selected from the group consisting of morphine, codeine, codeinone, thebaine, oripavine, reticuline, (S)-laudanine and laudanosine.

45. Plant having altered alkaloid biosynthesis, said plant containing in its genetic material, an exogenous nucleic acid comprising or consisting of an (S)-N-methylcoclaurine 3′-hydroxylase gene, or a derivative thereof.

46-51. (canceled)

52. Nucleic acid encoding (S)-N-methylcoclaurine 3′-hydroxylase having the amino acid sequence illustrated in FIG. 6 (SEQ. ID. N^{o} 2).

53-55. (canceled)
